(19) United States
(12) Patent Application Publication
(10) Pub. No.: US 2026/0049927 A1
(43) Pub. Date: Feb. 19, 2026

(54) ANALYZER FOR AUTOMATICALLY DETECTING AND QUANTIFYING TETRAKIS(HYDROXYMETHYL)PHOSPHONIUM SULFATE IN BIOCIDE PRODUCTS

(71) Applicant: Saudi Arabian Oil Company, Dhahran (SA)

(72) Inventors: Xiangyang Zhu, Dhahran (SA); Mazen A. Al-Saleh, Al Khobar (SA); Fahad N. Al-Abeedi, Dammam (SA); Alexander Grigoryan, Dhahran (SA); Juan F. Hernandez Montiel, Al Khobar (SA)

(73) Assignee: Saudi Arabian Oil Company, Dhahran (SA)

(21) Appl. No.: 18/807,334

(22) Filed: Aug. 16, 2024

(51) Int. Cl. G01N 21/31; B01L 3/00; G01N 21/78; G01N 21/85; G01N 33/18; G16C 20/30

(52) U.S. Cl. CPC G01N 21/3151; B01L 3/502; G01N 21/78; G01N 21/85; G01N 33/184; G16C 20/30; B01L 2200/0684; B01L 2200/16; B01L 2300/047; B01L 2300/0663; B01L 2300/0681; B01L 2300/0877; B01L 2300/1805; B01L 2300/1894; B01L 2400/0487; B01L 2400/0622

(57) ABSTRACT

An analyzer is disclosed for detecting and quantifying a tetrakis(hydroxymethyl)phosphonium sulfate (THPS) composition in a water sample. The analyzer includes a peristaltic pump and a three-way valve, where the peristaltic pump draws in a water sample that includes a THPS composition through the 3-way valve. The analyzer also includes a 6-way valve that receives the water sample and a potassium permanganate reagent, and a reaction coil in which the water sample and the KMnO₄ reagent are mixed and reacted. The analyzer further includes a debubbler that remove bubbles from the mixture; and a flow cell that receives the mixture from the debubbler, measures an intensity of KMnO₄ absorption in the mixture, normalizes the measured intensity by subtracting a background intensity, and determine a presence and a concentration of the THPS composition in the water sample.

### TECHNICAL FIELD

The present disclosure is generally related to a device for detecting and quantifying tetrakis(hydroxymethyl)phosphonium sulfate in water.

### BACKGROUND

Tetrakis(hydroxymethyl)phosphonium sulfate (THPS) is a primary component in biocide products commonly used in the oil and gas industry for microbial control in water systems. Biocidal performance generally depends on the concentration and contact time with microorganisms, and thus it is important to monitor biocide residual concentration closely in any water system to ensure microbial control.

THPS-based biocides are readily degradable and thus are generally considered environmentally friendly. Conventionally, THPS is detected and quantified using iodometric titration method using commercial test kits, such as Solvay TOLCIDE Biocides Test kit. However, in a large water pipeline networks, it is extremely challenging to collect water samples for biocide residual measurement at downstream locations of the pipeline network, especially in remote locations, after a batch treatment of the biocide product at an upstream location. This is due to the difficulties to estimate the biocide travel time in the large pipeline network because of the complexity of pipeline network (pressure, diameters, branches, etc.), working schedule, and daily operation changes and fluctuations (e.g., flow rate).

Accordingly, there is a need for effective field measurement tools for THPS-based biocides in water networks, particularly those in oil and gas facilities. The present application addresses these and other challenges.

### SUMMARY

In a first aspect, an analyzer for detecting and quantifying a tetrakis(hydroxymethyl)phosphonium sulfate (THPS) composition in a water sample is provided. The analyzer includes a peristaltic pump and a three-way valve operatively connected to the peristaltic pump, wherein the peristaltic pump is configured to draw in a water sample comprising a THPS composition through the 3-way valve. The analyzer also includes a six-way valve operatively connected to the three-way valve via the peristaltic pump, wherein the six-way valve is configured to receive: a) the water sample from the 3-way valve via a first inlet and b) a potassium permanganate (KMnO₄) reagent via a second inlet. The analyzer further includes a reaction coil operatively connected to the 6-way valve and configured to receive the water sample and the KMnO₄ reagent, wherein the water sample and the KMnO₄ reagent are mixed and reacted in the reaction coil; and a debubbler operatively connected to reaction coil and configured to receive the mixture of the water sample and the KMnO₄ reagent and remove bubbles from the mixture. The analyzer also includes a flow cell in a Z configuration comprising a 525 nm LED and a 650 nm LED, wherein the flow cell is configured to: receive the mixture from the debubbler; measure an intensity of KMnO₄ absorption in the mixture at a wavelength of 525 nm; normalize the measured intensity by subtracting a background intensity at a wavelength of 650 nm; and determine a presence and a concentration of the THPS composition in the water sample.

In another aspect, a correlation of the KMnO₄ absorption and the concentration of the THPS composition is based on the equation:

$$Y = \frac{1}{a + bX} ,$$

wherein Y is the peak height of absorption signal, X is the concentration of the THPS composition in the water sample, and a and b are calibration factors determined using a 2-point calibration procedure.

In a further aspect, to perform the 2-point calibration procedure the flow cell is configured to: measure an intensity of KMnO₄ absorption at a wavelength of 525 nm in a mixture of a water sample without the THPS composition and the KMnO₄ reagent to determine calibration factor a, wherein

$$a = \frac{1}{PH}$$

and PH is the peak height of the absorption signal; and measure an intensity of KMnO₄ absorption at a wavelength of 525 nm in a mixture of a water sample with a known concentration c of the THPS composition and the KMnO₄ reagent to determine calibration factor b, wherein

$$b = \frac{\frac{1}{PH} - a}{c} .$$

In another aspect, the three-way valve comprises a water sample inlet for receiving the water sample, a calibration inlet for a calibration fluid, and an outlet 113 configured to direct the water sample or calibration fluid to the remaining components of the analyzer.

In another aspect, the analyzer further comprises a reagent injector configured to inject the potassium permanganate reagent into the 6-way valve.

In another aspect, the water sample is a calibration fluid, and wherein when the 3-way valve receives a calibration fluid, the analyzer is configured to perform a calibration cycle.

In another aspect, the analyzer has a dynamic detection range for the THPS composition of approximately 80-2000 ppm.

In another aspect, the THPS composition is a biocide composition and wherein the water is seawater.

In another aspect, the analyzer further comprises a heater operatively connected to the reaction coil, wherein the heater is configured to maintain the reaction coil at a temperature in the range of approximately 35-45° C. In a further aspect, the heater is configured to maintain the reaction coil at approximately 40° C.

In another aspect, the analyzer further comprises a cooling plate configured to cool a container comprising the KMnO₄ reagent, wherein the cooling plate is configured to maintain the container at a temperature in the range of approximately 2-8° C.

In another aspect, the measurement accuracy of the concentration of the THPS composition is within ±20% of the actual THPS concentration.

In another aspect, the analyzer is an online analyzer operatively connected to a water system and configured to perform real-time measurements of water samples from the water system. In a further aspect, the analyzer is configured to automatically switch from a slug mode to a non-slug mode if the measured concentration of the THPS composition is below a first predetermined value and to switch from non-slug mode to slug mode if the measured concentration of the THPS composition is above a second predetermined value.

In another aspect, the analyzer further comprises: a filter device operatively connected to the water system via tubing that receives the water sample from the water system, wherein the filter device is configured to remove debris and bubbles from the water sample; and an intermediate sample vial operatively connected to the three-way valve and configured to receive the water sample after passing through the filter device and further remove bubbles in the water sample. The filter device and the intermediate sample vial are located upstream of the three-way valve.

In a second aspect, an online analyzer operatively connected to a water system for detecting and quantifying a tetrakis(hydroxymethyl)phosphonium sulfate (THPS) composition in a water sample is provided. The analyzer comprises a cross-flow filter operatively connected to the water system via tubing that receives a water sample comprising a THPS composition from the water system, wherein the filter device is configured to remove debris and bubbles from the water sample, and a piston pump configured to draw in the water sample from the water system through the cross-flow filter. The analyzer also includes a reaction vessel operatively connected to the piston pump and configured to receive the water sample via the piston pump and a potassium permanganate (KMnO₄) reagent from a reagent supply, wherein the water sample and the KMnO₄ reagent are mixed and reacted in the reaction vessel and the reaction vessel is further configured to remove bubbles from the water sample and KMnO₄ reagent mixture. The analyzer further includes a flow cell in a Z configuration comprising a 525 nm LED and a 650 nm LED, wherein the flow cell is configured to: receive the water sample and KMnO₄ reagent mixture from the reaction vessel; measure an intensity of KMnO₄ absorption in the mixture at a wavelength of 525 nm; normalize the measured intensity by subtracting a background intensity at a wavelength of 650 nm; and determine a presence and a concentration of the THPS composition in the water sample.

In another aspect, a correlation of the KMnO₄ absorption and the concentration of the THPS composition is based on the equation:

$$Y = \frac{1}{a + bX} ,$$

wherein Y is the peak height of absorption signal, X is the concentration of the THPS composition in the water sample, and a and b are calibration factors determined using a 2-point calibration procedure.

In a further aspect, to perform the 2-point calibration procedure the flow cell is configured to: measure an intensity of KMnO₄ absorption at a wavelength of 525 nm in a mixture of a water sample without the THPS composition and the KMnO₄ reagent to determine calibration factor a, wherein

$$a = \frac{1}{PH}$$

and PH is the peak height of the absorption signal; and measure an intensity of KMnO₄ absorption at a wavelength of 525 nm in a mixture of a water sample with a known concentration c of the THPS composition and the KMnO₄ reagent to determine calibration factor b, wherein

$$b = \frac{\frac{1}{PH} - a}{c} .$$

In another aspect, the analyzer has a dynamic detection range for the THPS composition of approximately 80-2000 ppm.

In another aspect, the measurement accuracy of the concentration of the THPS composition is within ±20% of the actual THPS concentration.

### BRIEF DESCRIPTION OF THE DRAWING FIGURES

FIGS. 1A-1C show diagrams of various aspects of an exemplary THPS analyzer in accordance with one or more embodiments.

FIG. 2 shows an exemplary calibration curve for THPS measurement based on a two-point calibration method in accordance with one or more embodiments.

FIG. 3 shows an exemplary THPS calibration curve based on five concentrations of THPS solutions (triplicate) in accordance with one or more embodiments.

FIGS. 4A-4C shows images of an exemplary THPS analyzer with the cabinet doors closed (FIG. 4A); the front door open (FIG. 4B) showing the liquid flow aspects of the analyzer system; and the back door open (FIG. 4C) showing the electronic aspects of the analyzer system, in accordance with one or more embodiments.

FIG. 4D displays an image of an exemplary debubbler in accordance with one or more embodiments.

FIG. 5 displays a schematic view of potassium permanganate injection to the stream of the water sample or calibration solution before (left diagram) and after (right diagram) the reaction in a knitted reaction coil (center image) in accordance with one or more embodiments.

FIG. 6 displays an image of exemplary main parameter settings in a user interface of the THPS analyzer in accordance with one or more embodiments.

FIG. 7 shows an exemplary THPS-based biocide measurement cycle in accordance with one or more embodiments.

FIGS. 8A-8B displays graphs of THPS-based biocide slug measurements in seawater samples with 0 ppm of biocide (FIG. 8A) and 556 ppm of the biocide BIOC31705A (FIG. 8B) as determined by the THPS analyzer in accordance with one or more embodiments.

FIG. 9 displays a calibration curve and dynamic detection range for a THPS-based biocide (BT4535) as determined by the THPS analyzer in accordance with one or more embodiments.

FIG. 10 displays the results of two-point calibration and acceptance tests for a THPS-based biocide (BT4535) solutions prepared at low, medium, and high concentrations in accordance with one or more embodiments.

FIG. 11 displays a calibration curve and dynamic detection range for a THPS-based biocide (RO-IM-B276) as determined by the THPS analyzer in accordance with one or more embodiments.

FIG. 12 displays the results of two-point calibration and acceptance tests for a THPS-based biocide (RO-IM-B276) solutions prepared at low, medium, and high concentrations in accordance with one or more embodiments.

FIG. 13 displays an exemplary connection of the THPS analyzer and a water sampling port (e.g., seawater sampling port) connected to a water system in accordance with one or more embodiments.

FIGS. 14A-14C display exemplary slug measurement curves for seawater samples with various concentrations of THPS-based biocide (BIOC31705A), 0 ppm (FIG. 14A), 278 ppm (FIG. 14B), and 1500 ppm (FIG. 14C), in accordance with one or more embodiments.

FIG. 15 displays the results of THPS-based biocide (BIOC31705A) slug detection measurements at a water supply plant in accordance with one or more embodiments.

FIG. 16 displays the results of another THPS-based biocide (BIOC31705A) slug detection measurements at a water supply plant in accordance with one or more embodiments.

FIG. 17 displays a THPS analyzer comprising additional features for the liquid handling aspects of the analyzer in accordance with one or more embodiments.

FIG. 18 is a block diagram illustrating an exemplary configuration of an exemplary computing device operatively connected to the THPS analyzer in accordance with one or more embodiments.

### DETAILED DESCRIPTION OF CERTAIN EMBODIMENTS

By way of overview and introduction, the present application discloses an analyzer for detecting and quantifying tetrakis(hydroxymethyl)phosphonium sulfate (THPS)-based compounds, such as biocides, in water samples. In certain embodiments, the water samples are collected from seawater pipelines. The analyzer allows for online (e.g., operatively connected to the water system) and real-time detection and monitoring of THPS-based biocide residuals water systems. In one or more embodiments, the analyzer has dynamic detection range of approximately 80-2000 ppm of a THPS-based biocide formulation (or approximately 20-800 ppm of THPS). In one or more embodiments, the measurement repeatability is less than approximately 10% variation in terms of relative standard deviation (RSD), and the measurement accuracy is within approximately ±20% of the actual THPS-based biocide concentration. The present THPS analyzer also provides improved ruggedness and resilience in large water treatment and injection systems, as well as improved liquid handling.

The present THPS analyzer is effective at real-time detection of THPS-based biocides and monitoring of the biocide residual concentration in a water system (e.g., large pipeline network), especially in remote injection wells, in order to ensure effective biocide treatment and maintain good microbial control in the water system. Specifically, in one or more embodiments, the THPS analyzer can provide real-time monitoring of residue concentrations of THPS biocides in downstream locations of a large pipeline network.

These and other aspects of the present analyzer and associated methods are described in further detail below with reference to the accompanied drawing figures, in which one or more illustrated embodiments and/or arrangements of the apparatus and methods are shown. The apparatus and methods of the present application are not limited in anyway to the illustrated embodiment and/or arrangement. It should be understood that the apparatus and methods as shown in the accompanying figures are merely exemplary of the apparatus and methods of the present application, which can be embodied in various forms as appreciated by one skilled in the art. Therefore, it is to be understood that any structural and functional details disclosed herein are not to be interpreted as limiting the present apparatus and methods, but rather are provided as a representative embodiment and/or arrangement for teaching one skilled in the art one or more ways to implement the present apparatus and methods. It should be understood that, as used in the present application, the term “approximately” when used in conjunction with a number refers to any number within 5% of the referenced number, including the referenced number.

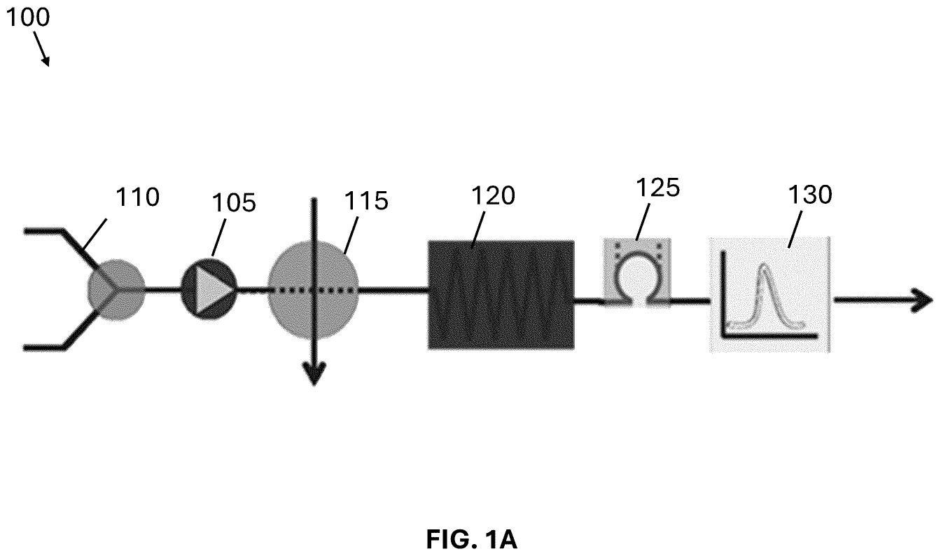

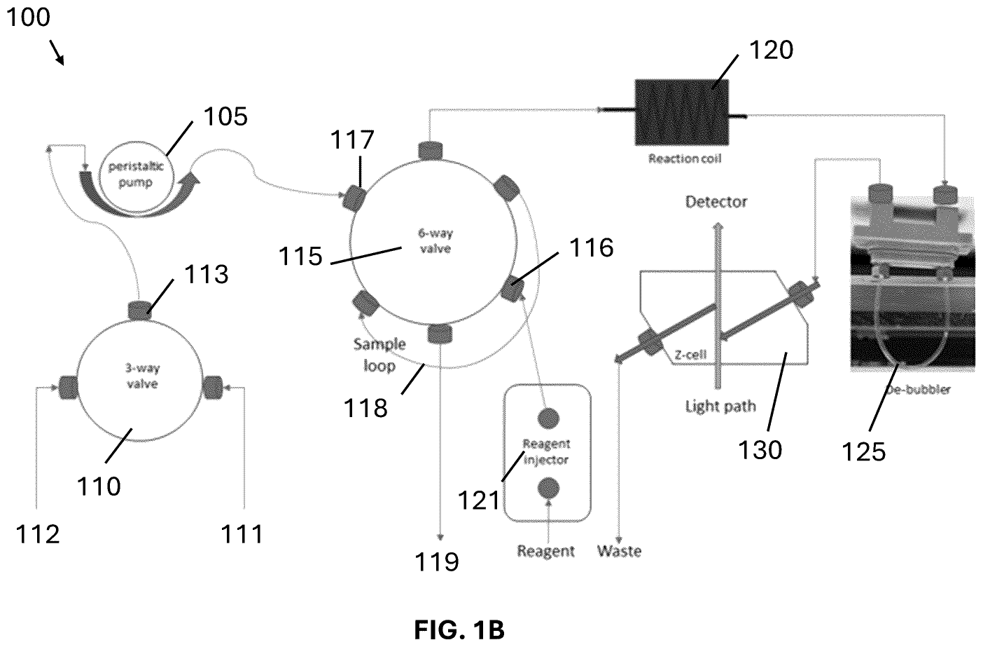

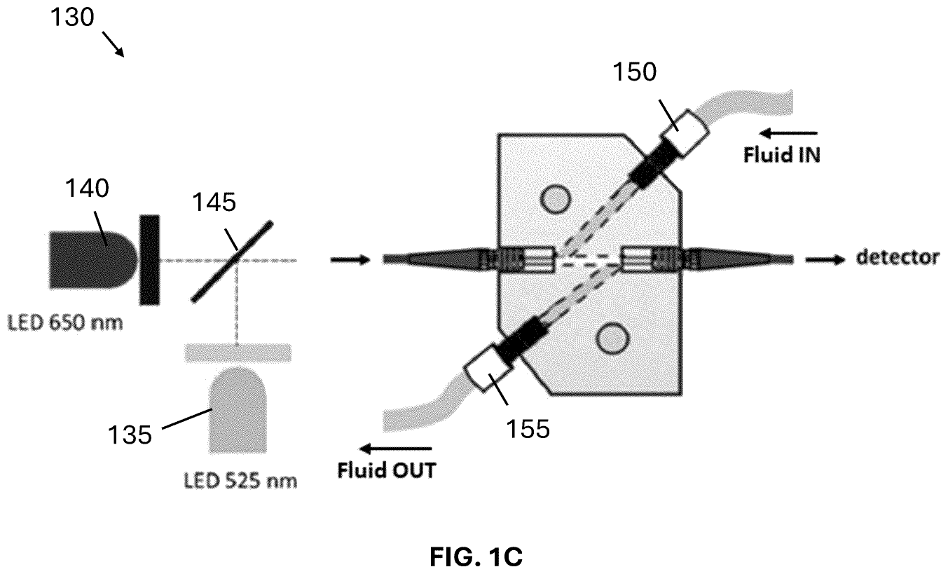

FIGS. 1A-1C show diagrams of various aspects of an exemplary THPS analyzer in accordance with one or more embodiments. With reference now to FIGS. 1A-1B, the THPS analyzer 100 includes a peristaltic pump 105. The pump 105 draws the water sample into the analyzer 100 via a three-way valve 110. In one or more embodiments, the pump 105 can be configured to draw in a calibration fluid into the analyzer 100 via the three-way valve 110. In certain embodiments, as exemplified in FIG. 1B, the three-way valve 110 can comprise a first inlet 111 for the water sample and a second inlet 112 for the calibration fluid, as well as an outlet 113 to direct the water sample or calibration fluid to other components of the analyzer 100. In one or more embodiments, the calibration fluid comprises of a THPS-based composition (e.g., THPS-based biocide formulation) with a known concentration.

The analyzer 100 also include a six-way valve or loop injector 115 that is operatively connected to the three-way valve 110 via the pump 105. During a calibration cycle or a sample measurement cycle, the peristaltic pump 105 draws a water sample or calibration fluid via the three-way valve 110 to the six-way valve or loop injector 115.

As exemplified in FIG. 1B, the loop injector 115 is also configured to receive a potassium permanganate solution reagent via a reagent injector 121 (or other mechanism) through an inlet 116 and the water sample or calibration fluid from the three-way valve through another inlet 117, and deliver the reagent to the water sample or calibration fluid. The loop injector 115 can also include sample loop 118 and a drain 119. In one or more embodiments, the reagent can be held in a container that is part of or operatively connected to the analyzer 100.

With continued reference to FIGS. 1A-1B, the analyzer 100 also includes a mixing coil or reaction coil 120 for mixing the reagent with the water sample or calibration solution. The loop injector 115 is operatively connected to the mixing/reaction coil 120 and configured to deliver the reagent and the water sample (or calibration solution) to the reaction coil 120. The reagent and the water sample (or calibration solution) are thoroughly mixed in the reaction coil 120 to cause a chemical reaction. Specifically, the THPS (if present) in the water sample or calibration solution, will react with the potassium permanganate (KMnO₄) solution (reagent), as explained in further detail below.

The mixture of the water sample (or calibration solution) and the reagent then flow to a debubbler 125, which is positioned downstream of the reaction coil 120 and is configured to remove any bubbles from the chemical reaction mixture and the de-pressurized water sample.

The analyzer 100 also includes an optical detector 130 operatively connected to the debubbler 125 and configured to receive the reaction mixture from the debubbler 125. In one or more embodiments, the optical detector 130 comprises a Z-flow cell configuration. As exemplified in FIG. 1C, in one or more embodiments, the Z-flow cell is a typical Z-flow cell having a Z-shaped fluidic path, which allows continuous flow up through the flow cell, and minimizes any residual bubble entrapment. The optical detector 130 having a Z-flow cell configuration comprises a 525-nm light-emitting diode (LED) 135 and a reference 650-nm LED 140 are arranged perpendicularly and directed at a dichroic mirror 145. In one or more embodiments, the Z-flow cell configuration can include SMA connectors for attachment to optical configuration and a data processor (e.g., microcontroller), for example. In a preferred optical configuration for the Z-flow cell shown in FIG. 1C, the reaction mixture of the water sample and the KMnO₄ enter the Z-flow cell via inlet 150, the two LED light 135 and 140 with specific wavelengths (525 nm and 650 nm, respectively) pass through the fluid (e.g., KMnO₄ and water sample mixture) and the absorption can be measured by an optical detector (not shown). The maximum absorption of potassium permanganate is measured at 525 nm and 650 nm is used as the reference measurement. The net absorption signal (peak height, PH) is calculated by subtracting the baseline signal from the maximum absorptions. The 650 nm correction improves the resolution at low THPS concentration ranges (i.e., high KMnO₄ absorption).

In one or more embodiments, the Z-flow cell has an absorption path length of approximately 1 cm. When the absorption of the KMnO₄ is collected by the flow cell, a signal is transported via an optical cable (optical fiber) to a data processor to process the analogue signals and provide pulses in order to measure the absorption of the KMnO₄. Specifically, the optical detector 130 is configured to measure an intensity of KMnO₄ absorption in the reaction mixture at a wavelength of 525 nm, normalize the measured intensity by subtracting a background intensity at a wavelength of 650 nm; and determine a presence and a concentration of the THPS composition in the water sample. After the absorption of the KMnO₄ has been measured and the concentration of THPS in the water sample has been determined, the mixture of the THPS water sample and the KMnO₄ is passed out of the flow cell via outlet 155 can be disposed of as waste.

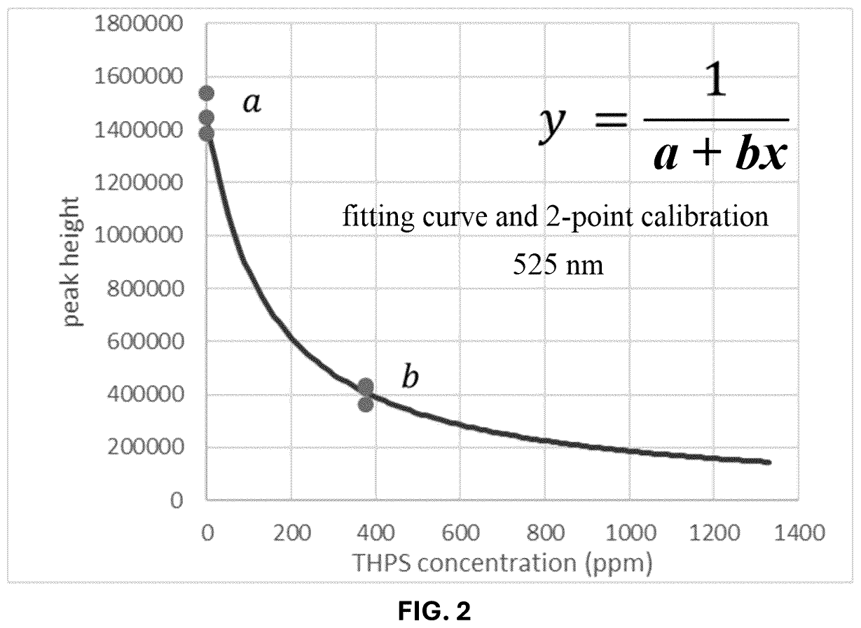

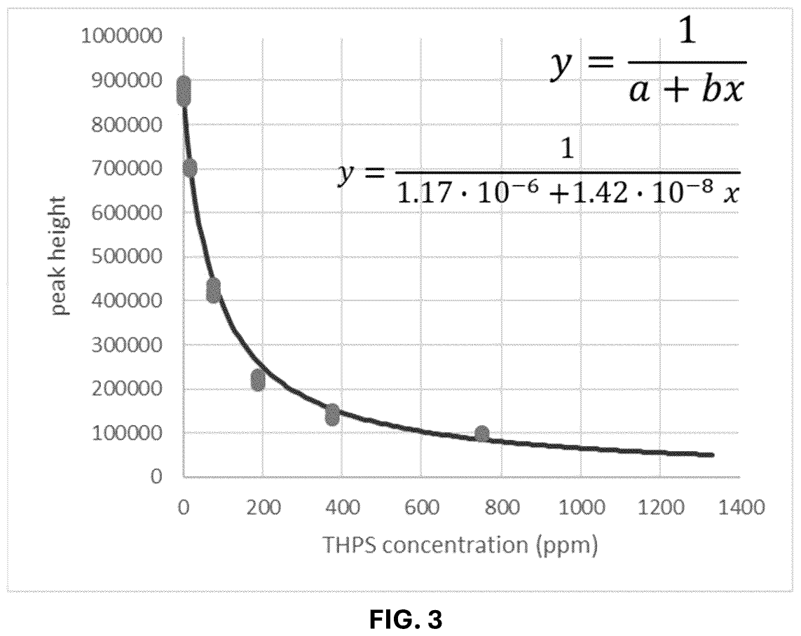

In one or more embodiments, the determination of the presence and concentration of THPS in the sample is based on the absorption measurement of potassium permanganate (KMnO₄) solution (pH 5.0) at 525 nm for THPS detection. THPS reacts with potassium permanganate to decolorize the purple potassium permanganate solution as a function of the THPS concentration. Specifically, in one or more embodiments, approximately 2 moles of THPS decolorizes 1 mole of KMnO₄. The color change can be measured by optical absorption at the maximum wavelength 525 nm. By measuring the absorption change of the mixture following the reaction, the THPS concentration can be determined. The correlation of absorption of permanganate solution and THPS concentrations fits into the following equation:

$$Y = \frac{1}{a + bX} ,$$

where Y is the peak height of absorption signal; X is the concentration of THPS biocide (ppm); and the calibration factors a and b are determined using a 2-point calibration procedure. An exemplary calibration curve for THPS measurement based on two-point calibration is shown in FIG. 2. While flowing through the Z-cell, the absorption maximum of permanganate is measured at 525 nm. The net absorption peak signals at 0 ppm (1st point) and another known concentration (2nd point) of the THPS-based composition (e.g., biocide) are used to calculate the calibration factors a and b, and calibration equation (FIG. 2). For unknown samples, the net absorption peak signals are used to determine the concentration of THPS-based composition in the water sample using the calibration equation. An exemplary THPS calibration curve based on five THPS concentrations (and a blank) is shown in FIG. 3.

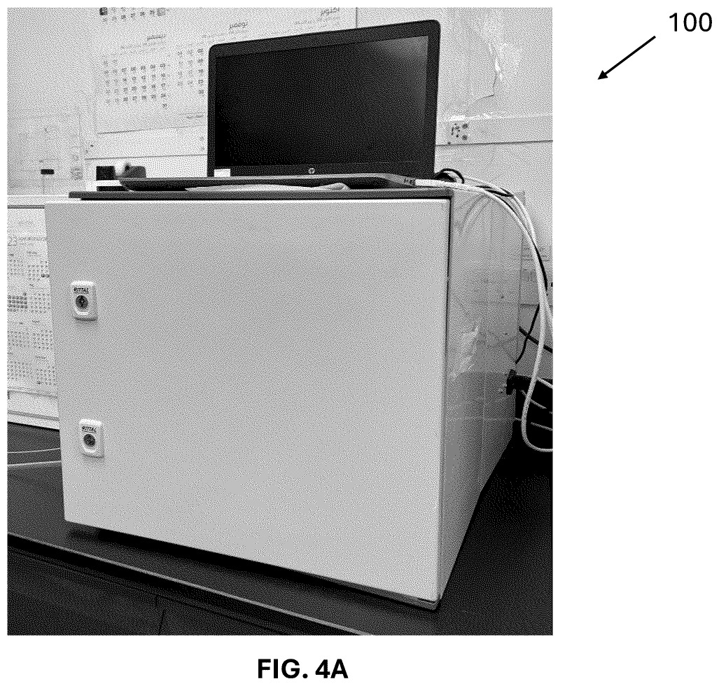

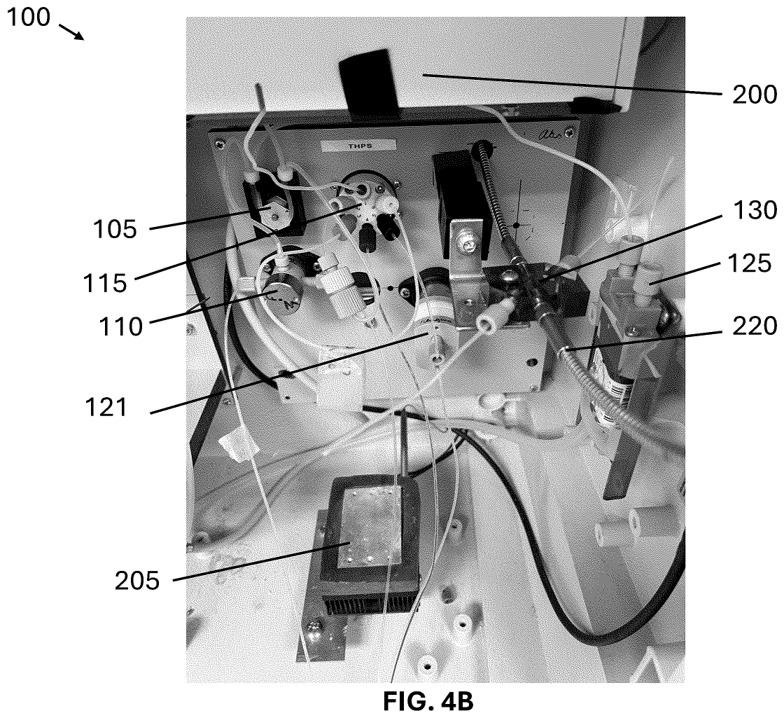

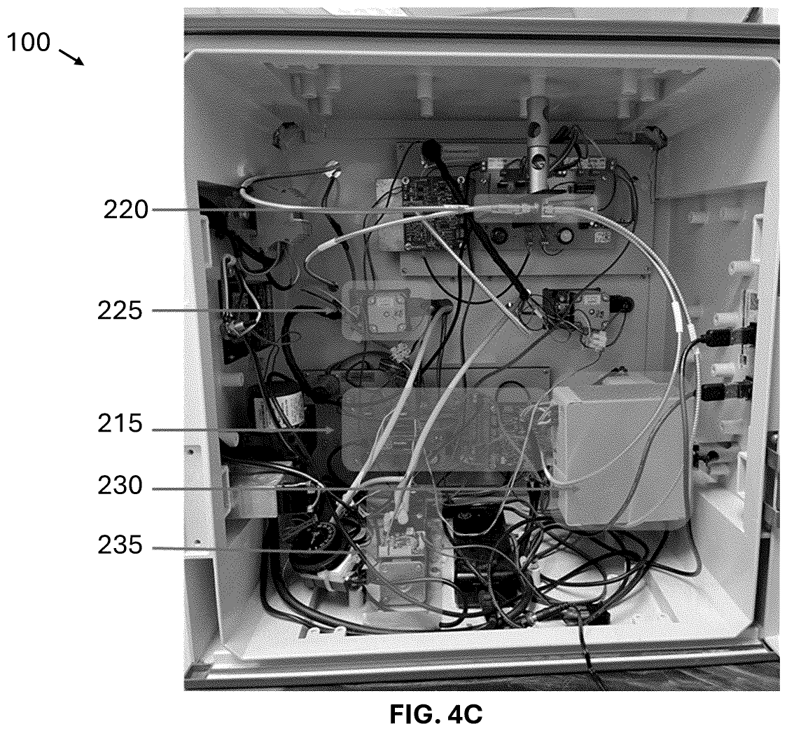

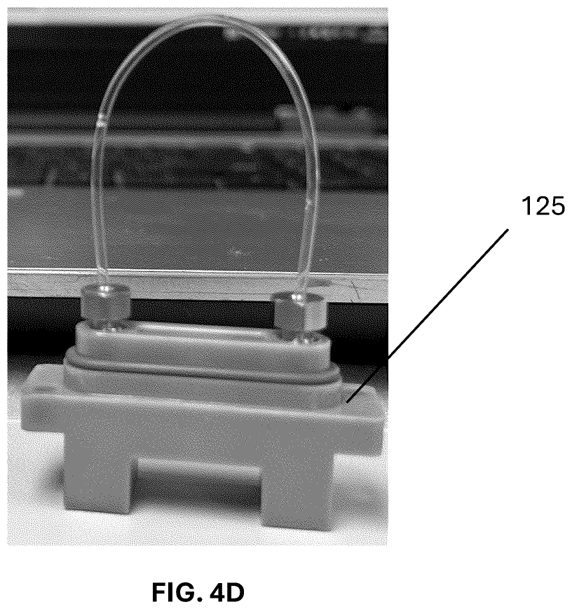

In one or more embodiments, the THPS analyzer of the present application can be constructed using an enclosure or cabinet for protection and temperature control. For example, in one or more embodiments the THPS analyzer can be constructed using mounted cabinets that, in certain instances, can be arranged back-to-back. FIGS. 4A-4C show images of an exemplary THPS analyzer with the cabinet doors closed (FIG. 4A), the front door open (FIG. 4B) showing the liquid flow aspects of the analyzer system, and the back door open (FIG. 4C) showing the electronic aspects of the analyzer system.

As exemplified in FIG. 4B, the front cabinet houses the liquid flow aspects of the system, including the processing fluids (e.g., water sample or calibration fluid, and reagent), the detector 130 (including the flow cell), reaction coil 120, debubbler 125, 3-way and 6-way valves (110 and 115, respectively), reagent injector 121, and peristaltic pump 105. In one or more embodiments, the liquid flow aspects of the analyzer can further include a heater 200 that encompasses the reaction coil 120. In one or more embodiments, the heater 200 can be configured to be maintained at a temperature in the range of approximately 35-45° C. In at least one embodiment, the heater 200 can be configured to be maintained at a temperature of approximately 40° C. In one or more embodiments, the liquid flow aspects of the analyzer can further include a cooling plate 205 for configured to cool the container comprising the reagent (e.g., potassium permanganate). The cooling plate 205 can be configured to be maintained at a temperature of approximately 4 C. In one or more embodiments, the cooling plate 205 can be configured to be maintained at a temperature in the range of approximately 2-8° C. for refrigeration of the reagent.

As exemplified in FIG. 4C, the back cabinet can include the electronic aspects of the analyzer, including the control printed circuit boards (PCBs) 215, the optical fiber 220, the engine 225 for the peristaltic pump, a power supple 230, and a pump 235 for the debubbler. An image of an exemplary debubbler 125 is shown in FIG. 4D in accordance with one or more embodiments.

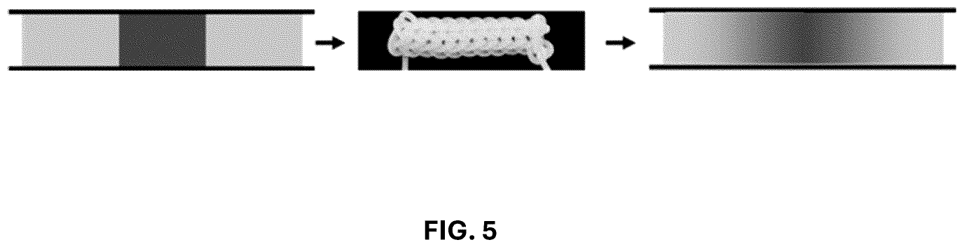

As previously mentioned, during a calibration cycle or a sample measurement cycle, the peristaltic pump draws in the calibration solution (e.g., solution having known concentration of THPS) or the water sample via a 3-way valve to the 6-way valve. The reagent (e.g., 1.2-1.5 mM KMnO₄ solution, pH 5.0) can then be injected into the calibration solution or water sample in the 6-way valve via an injector (e.g., micropump). For example, in at least one embodiment, a micropump can injects 50 μl of reagent into the THPS-containing water sample or calibration solution. FIG. 5 displays a schematic view of potassium permanganate injected into the stream of the calibration solution or water sample, before (left diagram) and after (right diagram) the mixture of the potassium permanganate and the water sample (or calibration solution) enter a knitted reaction coil (middle image). The calibration solution or THPS-containing water sample and the reagent are mixed thoroughly and react at 40° C. when they move through the reaction coil, which in certain embodiments can be approximately 1 meter in length. The mixed reaction fluid passes through the debubbler (e.g., 100 μl capacity, with −0.99 barg vacuum pump) for removing bubbles in the mixture. The de-gassed mixture then flows through the Z-cell of the detector to a waste container.

In one or more embodiments, the THPS analyzer of the present application is configured to operate on freshwater samples or salt water (seawater) samples. In one or more embodiments, the THPS analyzer is configured to operate on seawater samples having up to approximately 5.5% salinity. In at least one embodiment, the THPS analyzer is configured to operate on seawater samples having approximately 7-15% salinity.

In certain embodiments, the THPS analyzer is designed to operate under lab conditions for temperature control. For example, the THPS analyzer can be installed in the other locations (e.g., a remote area) with an enclosure and air conditioning.

In one or more embodiments, the water system that the THPS analyzer receives the water sample from is a pressurized water system, and thus the water pressure of the water sample needs to be regulated down (de-pressurized) to be at or near approximately 1 atm before entering the THPS analyzer.

Exemplary parameters for the THPS analyzer of the present application are shown in Table 1 below.

TABLE 1
Specification of prototype THPS analyzer.
Specifications of prototype THPS analyzer
Reagent

| | |
|---|---|
| Concentration | 1.2~1.5 mM KMnO₄ |
| | in 0.1M HCl |
| Reagent injection volume | 50 μl |
| Shelf life | At least one month at 4° C. |

Sampling

| | |
|---|---|
| Sample flow rate | 150 μl/min |
| Sample temperature | <40° C. |
| Sample pressure | normal pressure (1 atm) |
| Max particles | 0.2 μm |

Measurement

| | |
|---|---|
| Detection method | Absorbance |
| Wavelength | 525 nm/625 nm |
| Maximum measurement | 5 times per hour |
| frequency | |
| Calibrator* | THPS- based formulation |
| Detection range | 20-800 mg/L THPS |
| Repeatability (RSD) | <10% |
| Accuracy | ±20% at 20 ppm |
| | ±20% at 400 ppm |
| | ±20% at 800 ppm |
| Measurement time | 6 minutes |

Connectivity

| | |
|---|---|
| Communication | Serial over USB |
| Hardware Accessibility | Cabinet lockable |
| Software Accessibility | Optional password |

General

| | |
|---|---|
| Reagent + calibrator temperature | 4° C. |
| Debubbler/degasser | Yes |
| Power supply | 110-120 V/200-240 V/300 |
| | VA/50/60 Hz |
| Housing Material | ABS steel |
| Dimensions H × W × D | 50 × 30 × 60 cm |
| Weight | Typical 15 kg |

*Concentration of analyte (THPS) varies in the formulations.

### 2-Point Calibration Procedure

As mentioned above, the correlation of absorption of permanganate solution and THPS concentrations fits into the following equation:

$$Y = \frac{1}{a + bX} ,$$

where Y is the peak height of absorption signal; X is the concentration of THPS biocide (ppm); and the calibration factors a and b are determined using a 2-point calibration procedure.

In one or more embodiments, the THPS analyzer of the present application is connected to the source of the water sample or a line connected the water sample source, and water sample stream flows through the analyzer continuously. During a calibration cycle, the reagent (potassium permanganate) is injected into a water sample containing no THPS to form a mixture, and the mixture passes into the reaction coil for thorough mixing. The mixed solution passes the de-bubbler and then the Z-cell, where the absorption of the permanganate is measured at 525 nm. The measurement signals (peak height, PH) are calculated by subtracting the average baseline (BL) signals. The calibration factor a is then determined. This process to determine factor a is called CAL0:

$$a = \frac{1}{PH} .$$

Next, a water sample containing a known concentration of a THPS-based formulation (i.e., the calibration fluid) is prepared, e.g., a 625 ppm sample. The analyzer carries out the same measurement process, and obtains an absorption PH signal of permanganate. The calibration factor b is now determined. This process to determine factor b is called CAL1:

$$b = \frac{\frac{1}{PH} - a}{625} .$$

It should be understood that 625 ppm is an exemplary known concentration for the calibration fluid, and in other embodiments, the calibration fluid can have a different known concentration “c” of THPS, such that the generic equation for determining factor b is:

$$b = \frac{\frac{1}{PH} - a}{c} .$$

The calibration factors a and b can then be automatically updated via the data processor into software of the analyzer, and used in the future measurements of unknown water samples to determine the concentration of a THPS-based formulations in the samples.

### Measurement Procedure for Unknown Samples

After the analyzer has been calibrated and the factors a and b are determined, and the correlation of absorption PH and biocide concentration is established. When the sampling tube of the THPS analyzer is connected to an unknown sample (such as a sea water sample), it carries out the same measurement process as described above, and obtains an absorption PH. The analyzer, via the data processor can then determine the concentration of the THPS-based formulation (e.g., THPS-based biocide) in the unknown sample using PH and the correlation equation:

$$Conc . ( ppm ) = \frac{\frac{1}{PH} - a}{b}$$

Exemplary measurement conditions of the THPS analyzer are summarized below in Table 2. With the peristaltic pump, the sample flow rate is maintained at approximately 140-150 μl/min in accordance with one or more embodiments. In one or more embodiments, the reagent KMnO₄ solution is prepared at approximately 1.2-1.5 mM in 0.1M HCl, and approximately 50 μl reagent is injected into the sample stream by a micropump. A higher reagent concentration extends the upper detection limit, but lowers the resolution at low THPS concentrations. To counter the potential variation of the injection volume due to various reasons, the micropump can be set to perform more than 1 stroke of injection to fill the 50 μl sample loop, before the reagent is delivered to the sample stream. Mixing and reaction of THPS and the reagent takes place in the reaction coil at approximately 40° C. In one or more embodiments, the reaction coil can be approximately 1-2 meters in length. In at least one embodiment, the reaction coil can be approximately 1 meter in length. In one or more embodiments, the absorption of the permanganate solution is measured for approximately 6 minutes.

TABLE 2
Measurement conditions for prototype THPS analyzer.

| Parameter | value | |
|---|---|---|
| Reaction temperature | 40° | C. |
| Sample flow rate | 150 | μl/min |
| Injection volume (reagent) | 50 | μl |
| Reagent | 1.5 mM KMnO₄•HCl | |
| Length of reaction coil | 1 | m |
| Measurement time | 6 | min |
| Absorbance measurement | 525 | nm |

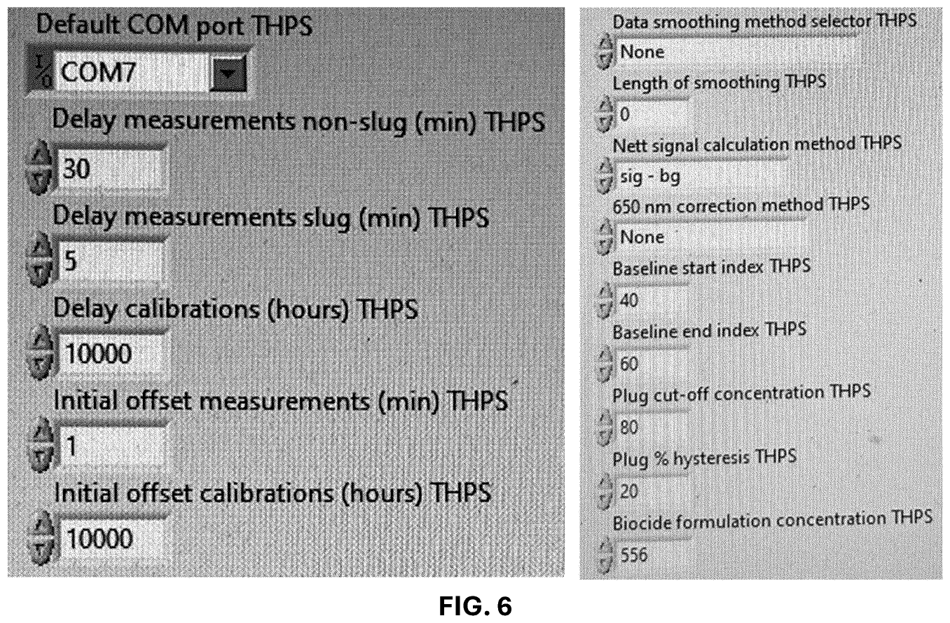

An example of the main parameter settings in the user interface of the present THPS analyzer in is shown in FIG. 6. For example, “delay measurements non-slug (min)” refers to the time between a measurement and the next measurement, when the system is not in slug mode. The term “delay measurements slug (min)” refers to the time between a measurement and the next measurement, when the system is in slug mode. The term “delay calibrations (hours)” refers to the time between a calibration and the next calibration. The term “initial offset measurements (min)” refers to the time between starting the program and the first measurement. The term “Initial offset calibrations (hours)” refers to the time between starting the program and the first calibration, and the term “baseline start & end index” refers to the indices over which to calculate the baseline. The term “plug cut-off concentration and hysteresis,” for example at 80 ppm and 20%, refers to the system being able to go from non-slug to slug mode when the concentration is above x ppm+y % (e.g., 96 ppm) or go from slug to non-slug mode when the concentration is below x ppm−y % (e.g., 64 ppm). Finally, the term “biocide formulation concentration” refers to concentration of the THPS-based formulation (e.g., biocide) in the calibration solution.

“Biocide batch treatment” is common in oil and gas industry and refers to biocide treatments in which the biocide is only injected into the water system at a pre-determined frequency (e.g., weekly) and for a pre-determined period of time (e.g., 2 hours). To prevent waste or depletion of the reagent, in certain embodiments the THPS analyzer of the present application can be configured automatically switch between “slug” and “non-slug” mode depending on the concentration of THPS biocide detected in the water system, where “slug” refers to the biocide batch treatment. For example, if the concentration of the THPS biocide is below a cut-off amount (e.g., 64 ppm), the THPS analyzer can be switch to non-slug mode (i.e. no biocide in the water system). Therefore, the analyzer will operate in non-slug mode with longer measurement intervals. On the other hand, if the THPS analyzer detects the THPS biocide above a cut-off amount (e.g., 96 ppm), the analyzer will operate in slug mode (i.e., biocide present in the water system). The analyzer will then measure the sample at shorter intervals in order to catch more data points before the slug passes the analyzer location.

In short, “slug” mode refers to when there is a biocide slug in the water system, and the THPS analyzer will measure more frequently to catch more data points before the slug passes. “Non-slug” mode means there is no biocide in the water system, and the THPS analyzer will measure less frequently to prevent unnecessary waste of the reagent. The THPS analyzer can switch the measurement mode automatically depending on the measured biocide concentration and the cut-off setting.

### Biocide Slug Measurement Cycle

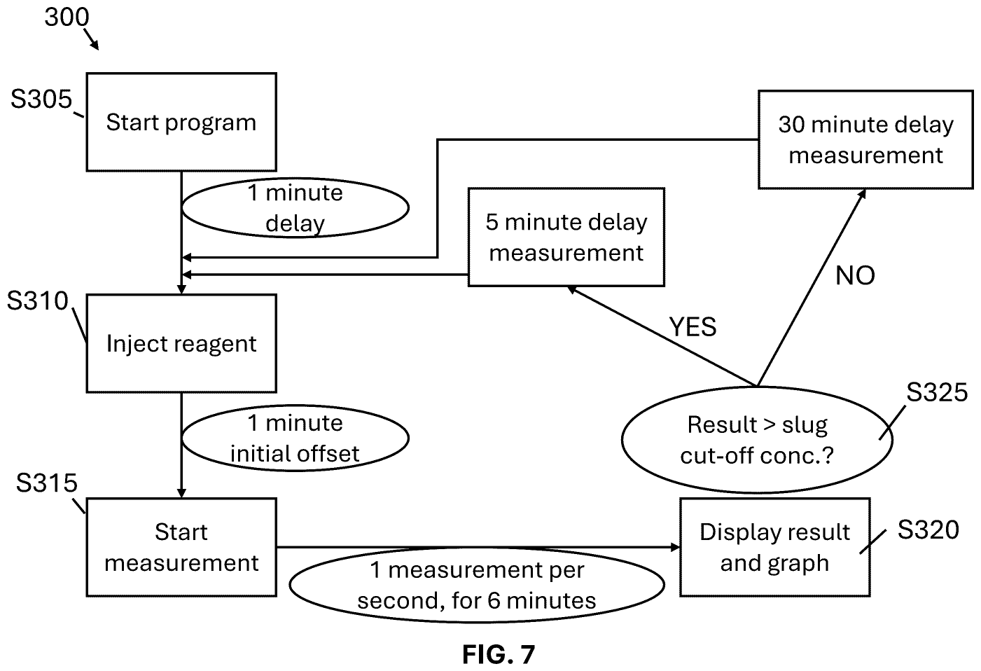

In accordance with one or more embodiments, an exemplary biocide slug measurement cycle is depicted in FIG. 7. Specifically, the flow diagram of FIG. 7 depicts a typical measurement cycle 300 for biocide slug in the source of the water sample. With reference to FIG. 7, at step S305 the cycle begins. At step S310, one min after starting the program, 50 μl reagent is injected into the sample stream. Then the analyzer waits for 1 min for the LED to stabilize and at step S315 starts the absorption measurement, one measurement per second for a total of six minutes. Then, at step S320 the biocide concentration is calculated using the correlation equation, and compares to the biocide plug cut-off concentration (e.g., 80 ppm±20%), and the result can be displayed.

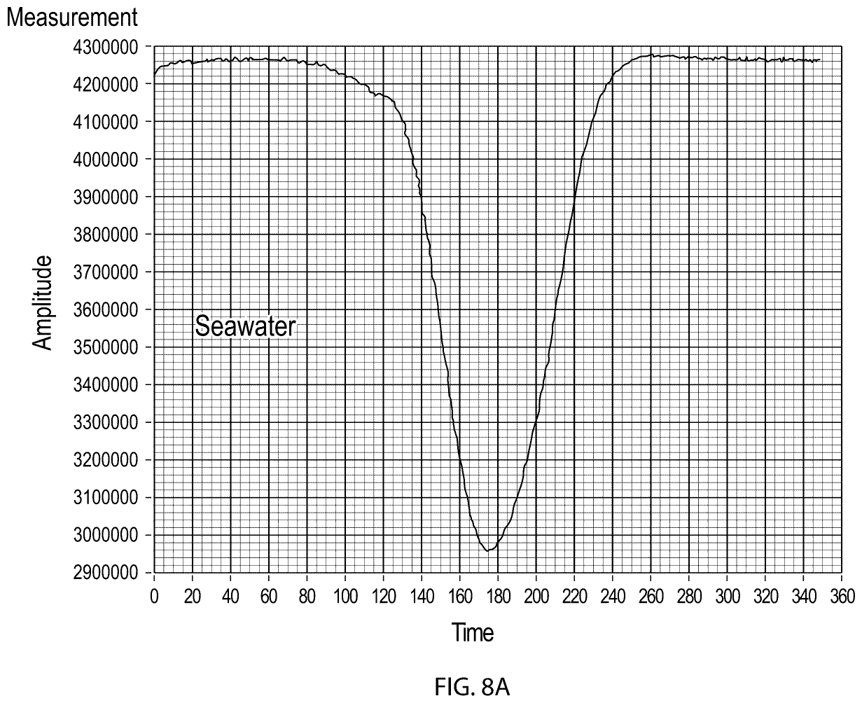

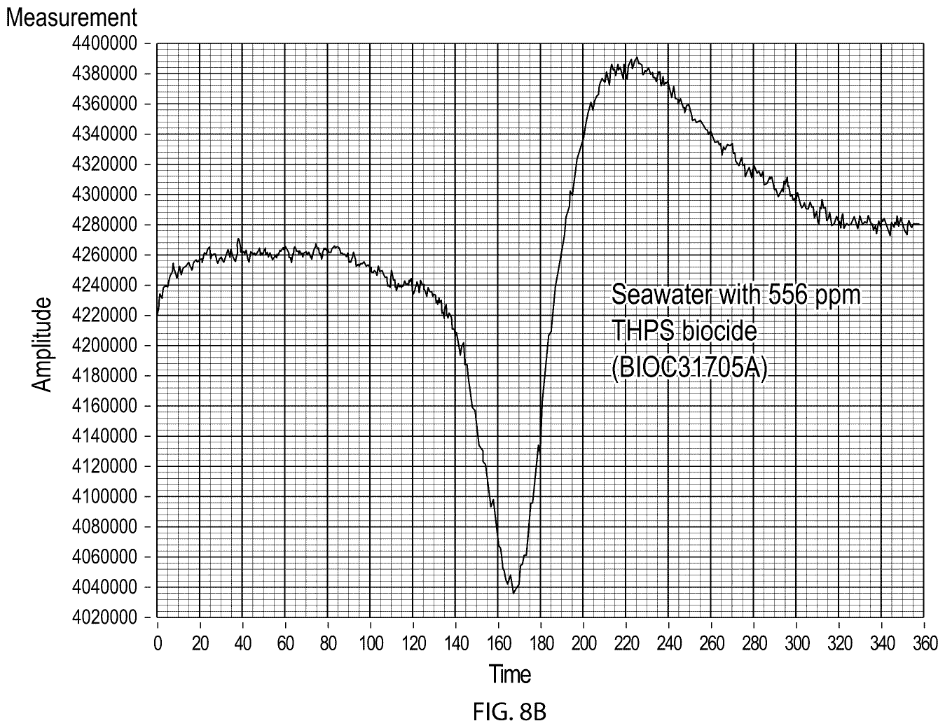

At step S325, the analyzer is configured to make a decision-specifically, to determine the analyzer's measurement mode (slug vs. non-slug mode)—for the next measurement. In one or more embodiments, the difference between these two modes is the delay time for the next measurement. Non-slug mode has a longer delay time (e.g., 30 min before next measurement), while the slug mode has a shorter delay time (e.g., 5 min) (see e.g., parameters FIG. 6). The operator can set a longer delay time to reduce the consumption of the reagent when the THPS-based biocide slug has not arrived at the detection site, for example. On the other hand, the operator can set a shorter delay time for the next measurement to allow the analyzer to take more measurements before the THPS-based biocide slug passes the detection site. A typical biocide slug measurement cycle takes 12 minutes (e.g., 1 minute initial offset, 6 minute measurement, and 5 minute delay for next measurement). The five minute delay allows the analyzer to clear the liquids in the flow system from the previous measurement, and therefore, minimize the carry-over signals, improving the quality of baseline signals—in other words, the signals will be more stable with less variation. Example graphs for THPS measurement on sea water samples containing 0 ppm and 556 ppm biocide (BIOC31705A), respectively, are shown in FIGS. 8A and 8B. Using the exemplary parameter settings in FIG. 6 as an example, the baseline signal is calculated by taking the average of measurement points from 40 to 60 seconds. The peak height (PH) signal is calculated by subtracting the baseline signal from the absorption maximum at 525 nm. It is noted that while these time values are optimized values for the THPS analyzer in certain embodiments, it should be understood that these values can be modified based on sample flow rate, the distance between sampling point and the flow cell, the size of de-bubbler, and delay time for next measurement, for example.

### Acceptance Tests of the THPS Analyzer

An acceptance test of the analyzer in accordance with one or more embodiments was performed to confirm hardware and software functionalities, and determine the dynamic measurement range, measurement repeatability (Relative Standard Deviation, RSD), and accuracy using commercial biocide products. The predefined criteria for the acceptance tests are <10% variation (RSD) for measurement repeatability and within ±20% for measurement accuracy when compared to the calculated value (see Table 3, below). Two THPS-based biocide products were selected for the acceptance tests (see Table 4, below). It is noted that the biocide RO-IM-B276 also contains 10-30% glutaraldehyde (GLUT).

TABLE 3
Predefined criteria for the acceptance
tests of prototype THPS analyzer.

| | | Prototype THPS |
|---|---|---|
| Criteria | | analyzer |
| Detection range | as active | 20-800 ppm THPS |
| | as product | 80-2000 ppm product |
| Repeatability | peak | <10% |
| (RSD) | height | |
| Accuracy (%) | as active | ±20% @ 20 ppm |
| | | ±20% @ 400 ppm |
| | | ±20% @ 800 ppm |
| | as product | ±20% @ 100 ppm |
| | | ±20% @ 1000 ppm |
| | | ±20% @ 2000 ppm |

TABLE 4
Biocide products used for acceptance tests.

| Biocide name | THPS | GLUT |
|---|---|---|
| BT4535 | 30-60% | |
| RO-IM-B276 | 10-30% | 10-30% |

The neat biocide products are considered as 100% or 1,000,000 ppm, and a series of dilutions was prepared in Qurayyah seawater (QSW) for the calibration curve, dynamic detection range, 2-point calibrations, and acceptance tests at low, medium and high concentrations (see Table 5, below). The concentrations of the active ingredient (THPS) in Table 5 were estimated based on the average of the active's ingredients concentration range (Table 4). Biocide BT4535 was diluted to 44-2222 ppm of biocide product (20-1000 ppm THPS active), and RO-IM-B276 diluted to 80-4000 ppm of biocide product (16-800 ppm THPS active). The concentration of the prepared biocide samples based on the product dilution is designated as the calculated concentration. The low-medium-high concentrations for the acceptance tests are in the range of 90 to 2000 ppm of biocide products (20-800 ppm of THPS active), which cover the concentration range in the biocide injection practices. The measurement conditions in the acceptance tests were shown in Table 2, above.

TABLE 5
Preparation of biocide products for various tests.

| | | product | THPS | RO-IM- | | product | THPS |
|---|---|---|---|---|---|---|---|
| BT4535 | DF* | ppm | ppm | B276 | DF | ppm | ppm |
| Calibration | 22,500 | 44 | 20 | Calibration | 12,500 | 80 | 16 |
| curve with | 9,000 | 111 | 50 | curve with | 5,000 | 200 | 40 |
| detection | 2,250 | 444 | 200 | detection | 1,250 | 800 | 160 |
| range | 900 | 1111 | 500 | range | 500 | 2,000 | 400 |
| | 450 | 2222 | 1000 | | 250 | 4,000 | 800 |
| 2-point Cal. | 1,800 | 556 | 250 | 2-point Cal. | 1,600 | 625 | 125 |
| Low | 11,250 | 89 | 40 | Low | 10,000 | 100 | 20 |
| Medium | 1,125 | 889 | 400 | Medium | 1,000 | 1,000 | 200 |
| High | 562.5 | 1778 | 800 | High | 500 | 2,000 | 400 |

*DF: dilution factor

Results of Acceptance Tests with Biocide Product BT4535

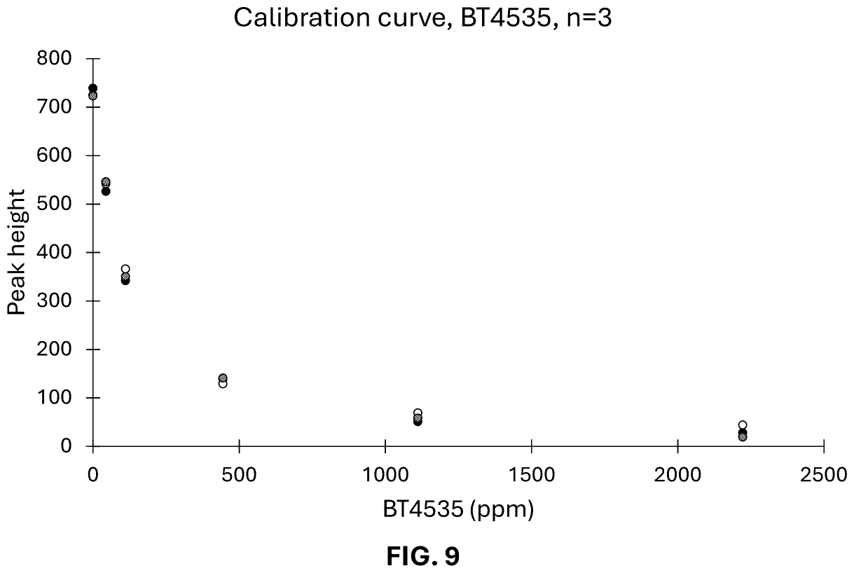

The dynamic detection range results for biocide BT4535 are shown in calibration curve of FIG. 9. The upper detection limit for BT4535 biocide is at least 2222 ppm, which is approximately 1000 ppm of THPS active (assuming the average THPS concentration in the formulation is 45%). The PH measurement repeatability (RSD) is between 1.2%-4.7% among the triplicate measurements at the concentration up to 444 ppm of BT4535 biocide (see Table 6, below), meeting the smaller than 10% criterion (see Table 3, above). At a higher concentration (1111 and 2222 ppm), the measurement variation is more than 10%; however, the later acceptance tests at medium and high concentrations (889 and 1778 ppm) showed the RSD below 2.2% (see Table 7, below).

TABLE 6
Peak Height (PH) measurement repeatability
(RSD) of BT4535 biocide.

| Calculated | Peak height (PH) | | |
|---|---|---|---|
| ppm, BT4535 | mean, n = 3 | Stdev | RSD (%) |
| 0 | 729.0 | 8.7 | 1.2% |
| 44 | 538.0 | 10.6 | 2.0% |
| 111 | 352.7 | 12.2 | 3.5% |
| 444 | 136.3 | 6.4 | 4.7% |
| 1111 | 59.3 | 9.1 | 15.3% |
| 2222 | 30.3 | 12.7 | 41.7% |

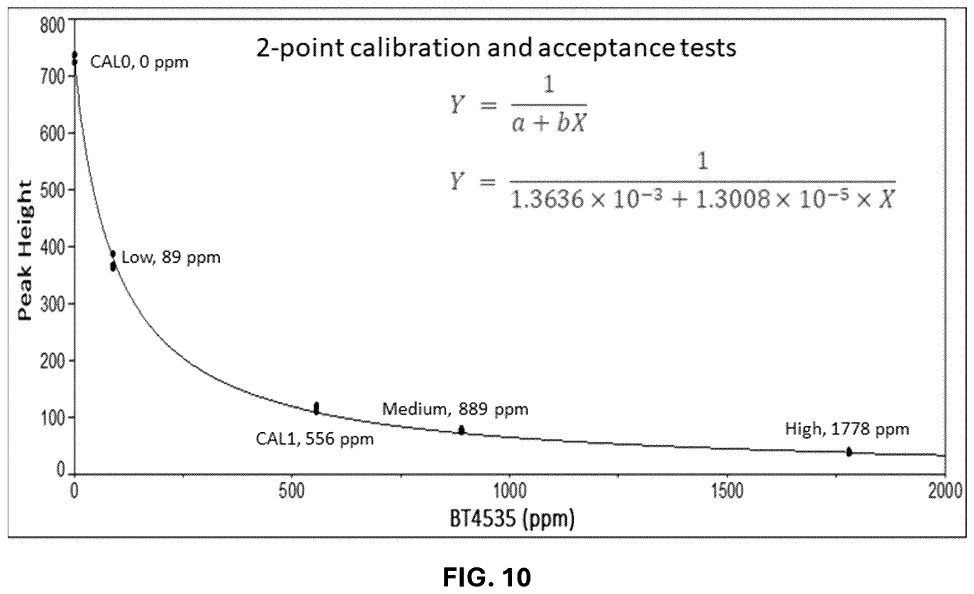

A 2-point (0 ppm and 556 ppm BT4535) calibration procedure was used to determine the factor a and b, and the calibration equation (FIG. 10). Three freshly prepared BT4535 solutions at low, medium, and high concentrations (89, 889, and 1778 ppm) were measured in triplicate using prototype THPS analyzer to determine the measurement repeatability (PH) and accuracy (ppm) (see FIG. 10; Table 7, below). The results in Table 7 showed that the measurement repeatability (RSD) of PH is between 3.7% and 6.6% among the triplicate measurements at low-medium-high concentration range, which meets the <10% criterion (see Table 3). The relative errors of the measured vs. calculated values (ppm) at low-medium-high concentration of BT4535 biocide were between −0.9% and 14.2%. This also meets the smaller than ±20% criterion (see, Table 3). In conclusion, the measurement of BT4535 biocide passed the predefined criteria for the acceptance tests.

TABLE 7
Measurement repeatability (PH) and accuracy of BT4535 biocide.

| | | | | | | Accuracy |
|---|---|---|---|---|---|---|
| | Calcu- | Peak height (PH) | | | | (%) |
| Acceptance | lated | mean, | | RSD | Measured | Measured |
| tests | ppm | n = 3 | stdev | (%) | ppm | vs calc. |
| 2-point cal. | 0 | 733.3 | 7.2 | 1.0% | | |
| | 556 | 116.3 | 5.0 | 4.3% | | |
| Low | 89 | 372.3 | 13.8 | 3.7% | 101.6 | 14.2% |
| Medium | 889 | 78.0 | 3.0 | 3.8% | 880.8 | −0.9% |
| High | 1778 | 40.0 | 2.6 | 6.6% | 1817.1 | 2.2% |

Results of Acceptance Tests with Biocide Product RO-IM-B276

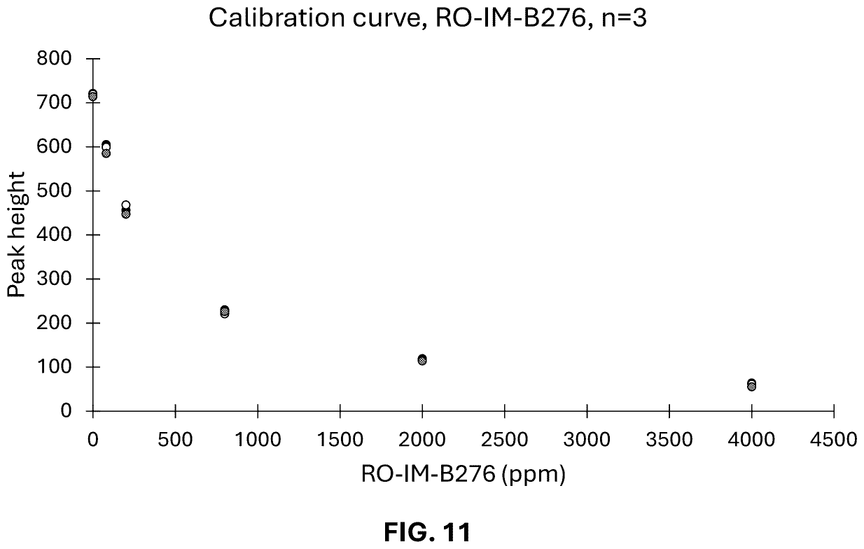

The dynamic detection range for biocide RO-IM-B276 is shown in the calibration curve of FIG. 11. The upper detection limit for RO-IM-B276 biocide is at least 4000 ppm, which is approximately 800 ppm of THPS active (assuming the average THPS concentration in the formulation is 20%). The PH measurement repeatability (RSD) is between 0.5%-7.6% among the triplicate measurements at the concentration range of up to 4000 ppm RO-IM-B276 biocide (Table 8, below), meeting the smaller than 10% criterion (see Table 3, above).

TABLE 8
PH measurement repeatability (RSD) of RO-IM-B276 biocide.

| Calculated | | | |
|---|---|---|---|
| ppm, | Peak height (PH) | | |
| RO-IM-B276 | mean, n = 3 | Stdev | RSD (%) |
| 0 | 718.0 | 3.6 | 0.5% |
| 80 | 596.3 | 10.3 | 1.7% |
| 200 | 457.0 | 10.5 | 2.3% |
| 800 | 225.7 | 4.5 | 2.0% |
| 2000 | 116.3 | 2.5 | 2.2% |
| 4000 | 60.0 | 4.6 | 7.6% |

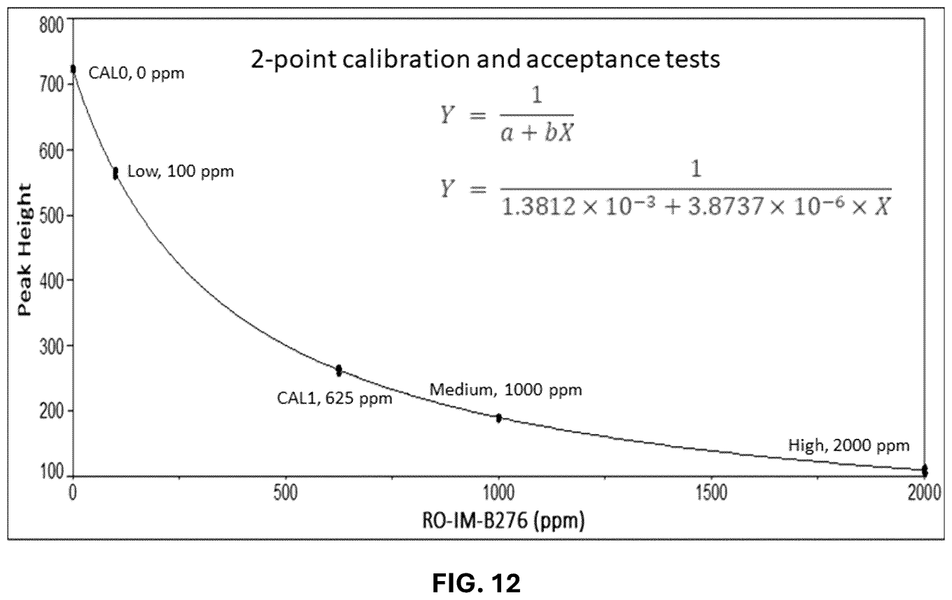

A 2-point calibration procedure (0 ppm and 625 ppm RO-IM-B276) was used to determine the factor a and b, and the calibration equation (see FIG. 12). Three freshly prepared RO-IM-B276 solutions at low, medium, and high concentrations (100, 1000, and 2000 ppm) were measured in triplicate using the THPS analyzer in accordance with one or more embodiments to determine the measurement repeatability (PH) and accuracy (see FIG. 12; Table 9, below).

The results in Table 9 showed that the measurement repeatability (RSD) of PH is between 0.8% and 4.3% among the triplicate measurements at low-medium-high concentration range, which meets the <10% criterion (see Table 3, above). The relative errors of the measured vs. calculated values (ppm) at low-medium-high concentration of RO-IM-B276 are within ±1.0%. This also meets the smaller than ±20% criterion (see Table 3, above). In conclusion, the measurement of biocide RO-IM-B276 biocide passed the predefined criteria for the acceptance tests. Again, it is noted that biocide RO-IM-B276 also contains 10-30% GLUT (see Table 4, above); but the results indicated that the presence of GLUT has no effect on the accurate measurement of THPS-based biocide.

TABLE 9
Measurement repeatability (PH) and
accuracy of RO-IM-B276 biocide.

| | | | | | | Accuracy |
|---|---|---|---|---|---|---|
| | Calcu- | Peak height (PH) | | | | (%) |
| Acceptance | lated | mean, | | RSD | Measured | Measured |
| tests | ppm | n = 3 | stdev | (%) | ppm | vs calc. |
| 2-point cal. | 0 | 724.0 | 1.7 | 0.2% | | |
| | 625 | 263.0 | 4.6 | 1.7% | | |
| Low | 100 | 566.0 | 4.4 | 0.8% | 99.5 | −0.5% |
| Medium | 1000 | 190.3 | 2.1 | 1.1% | 999.7 | 0.0% |
| High | 2000 | 108.7 | 4.7 | 4.3% | 2019.1 | 1.0% |

### Slug Detection Mode of Biocides

A biocide batch treatment is most common in oil and gas industry for various reasons. It is generally more cost-effective than continuous treatment in large oilfield systems over a long period of time. If applied properly, it can achieve the same goal as the continuous treatment—that is to bring microbial activities or counts under control in the target systems. In batch treatment, the biocide (250-1500 ppm) is injected into the target system at a pre-determined frequency (weekly being the most common) and for a pre-determined period of time (1-4 hours being the most common). In some situations, two biocides with different chemistries are used, alternating on weekly basis, for the purpose of avoiding or delaying the development of bacterial resistance or selection against the biocides.

To reduce the consumption of the reagents, and thereby also reduce the reagent refilling frequency, slug/non-slug detection mode was implemented in the THPS analyzer, in accordance with one or more embodiments. When the system water contains no biocide slug, the analyzer conducts the measurement cycles at longer intervals to save the reagents; however, when the analyzer detects the arrival of the biocide slug, it is configured to automatically switch to slug detection mode to measure the biocide residuals as many times as possible before the biocide slug passes the analyzer.

The determination between slug and non-slug measurements was implemented in the analyzer by means of a comparator function on the measured concentration of the product in ppm. For instance, the comparator function can be set at 80 ppm±20% (i.e. ±16 ppm) of the product (see, FIG. 6), which is called “plug cut-off concentration and hysteresis.” After each measurement the log output from the control software was closely monitored by the operator and the determination that were made by the analyzer were assessed for the following situations: 1) system in non-slug mode: stay in non-slug mode, if the measured concentration <64 ppm; 2) system in non-slug mode: go to slug mode, if the measured concentration >96 ppm; 3) system in slug mode: stay in slug mode, if the measured concentration >96 ppm; and 4) system in slug mode: go to non-slug mode, if the measured concentration <64 ppm.

Slug simulation was performed as part of acceptance tests (Table 10, below). While running the THPS analyzer with blank seawater, a sample of biocide product (BT4535) was added to the blank SW volume, aiming at an approximate product concentration of >96 ppm. Following the measurement of the spiked seawater (i.e., with BT4535), the sample supply was reverted to blank seawater and the next measurement was performed. During these alternations of biocide addition and removal, the system was monitored to determine whether the THPS analyzer would switch between slug and non-slug mode automatically based on the measured biocide concentration compared to the pre-set comparator function 80±16 ppm.

The results in Table 10 indicate that the determinations of the analyzer correctly followed the concentration changes that were measured.

TABLE 10
Example of slug/non-slug measurements of biocide BT4535.

| | Detected | Status/Change |
|---|---|---|
| Sample | product (ppm) | observed |
| SW/blank | <64 | non-slug |
| Added BT4535 | >96 | non-slug −> slug |
| Remained on BT4535 | >96 | slug |
| SW/blank | <64 | slug −> non-slug |
| Added BT4535 | >96 | non-slug −> slug |
| Remained on BT4535 | >96 | slug |
| SW/blank | <64 | slug −> non-slug |
| Added BT4535 | >96 | non-slug −> slug |
| Remained on BT4535 | >96 | slug |
| SW/blank | <64 | slug −> non-slug |
| SW/blank | <64 | non-slug |
| Added BT4535 | <64 | non-slug |
| Added BT4535 | >96 | non-slug −> slug |
| Added BT4535 | >96 | slug |
| SW/blank | <64 | non-slug |

### Conclusions of Acceptance Tests

The aim of the acceptance tests was to confirm functionalities of the analyzer, and determine the dynamic measurement range, measurement repeatability (RSD), and accuracy of the analyzer using commercial biocide products. The predefined criteria for the acceptance tests are <10% measurement variation (RSD) and ±20% for measurement accuracy when compared to the calculated value (Table 3). Table 11 summarizes the outcomes of acceptance tests using two THPS-based commercial biocide products. The measurements of both THPS-based biocides (BT4535 and RO-IM-B276) passed the predefined criteria for detection range, measurement repeatability (RSD), and accuracy. During the acceptance tests, it was concluded that the comparator logic performed as expected, i.e., the system switched from non-slug mode to slug mode and vice versa upon crossing the threshold of 80 ppm±20% biocide concentration. In other words, the acceptance tests demonstrated that the THPS analyzer can successfully switch between slug mode and non-slug mode measurements automatically depending on the measured THPS-based biocide concentration and the pre-set comparator parameters, saving the reagents and minimize the wear and tear of the analyzer parts.

TABLE 11
Summary of acceptance tests of THPS
analyzer for biocide products.

| | | | Repeat- | Accuracy | |
|---|---|---|---|---|---|
| | | Detection | ability | (%) | Accep- |
| | | range (ppm) | (RSD) | measured vs | tance |
| Biocide | Criteria | 80-2000 | <10% | calc. ±20% | tests |
| BT4535 | Calibration | 0-2222 | 1.2-41.7% | — | Pass |
| | curve | | | | |
| | Validation | 89/889/ | 3.7-6.6% | −0.9~14.2% | |
| | at L/M/H* | 1778 | | | |
| RO-IM- | Calibration | 0-4000 | 0.5-7.6% | — | Pass |
| B276 | curve | | | | |
| | Validation | 100/1000/ | 0.8-4.3% | −0.5~1.0% | |
| | at L/M/H | 2000 | | | |

*L/M/H: low-medium-high concentration of biocide product.

### Field Demonstration of THPS Analyzer

A field demonstration was conducted at a large seawater injection pipeline network. The analyzer was connected to the sampling port of a seawater shipping line at a water supply plant, 93 km downstream of the biocide injection site, where BIOC31705A (THPS-based) and BIOC31450A (GLUT-based) biocides were injected alternatively on weekly basis at 750 ppm for 90 minutes. The main ingredients for these two biocides are shown in Table 12 below. For the field demonstration, the THPS analyzer was used for online detection and quantification of THPS-based BIOC31705A residual when it arrives at the sampling port of the supply plant.

TABLE 12
The biocide products used in SW pipeline network.

| | | | Ethylene | |
|---|---|---|---|---|
| Biocide name | THPS | GLUT | alcohol | Methanol |
| BIOC31705A | 30-60% | | | |
| BIOC31450A | | 10-30% | 10-30% | 1-10% |

### THPS Analyzer Installation

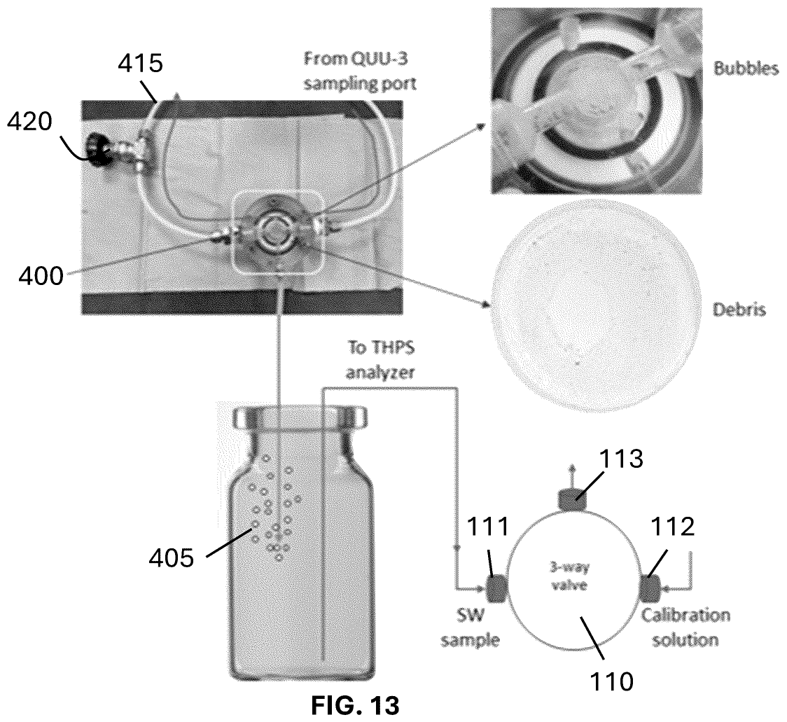

The THPS analyzer was installed in a field laboratory at the supply plant, 93 km downstream of the biocide injection site. It was connected to the sampling port of a 60″ seawater shipping line for automated detection and monitoring of biocide slug when it arrived at the supply plant. Two biocides (based on THPS or GLUT) were injected at the sea water treatment plant weekly and on alternating basis, meaning the THPS-based biocide would arrive at the supply plant every two weeks. The planned injection dosage was 750 ppm for 90 minutes. The connection of the seawater sampling port and the analyzer was configured as shown in FIG. 13 to address some challenges faced in the analyzer installation and demonstration.

The seawater shipping line is a pressurized system, and the seawater contains dissolved nitrogen gas used in a deaeration process. When the pressure is reduced from approximately 8 bar at the sampling port to a few millibars at the prototype THPS analyzer, a large amount of gas (bubbles) is released from the seawater stream, which interferes the measurement of absorption signals. In addition, the treated seawater contains 0.2 mg/L or more of total suspended solids (TSS), which can clog the analyzer's flow system or interfere with the absorption measurement.

FIG. 13 displays an exemplary connection of the THPS analyzer and a water sampling port (e.g., seawater sampling port) connected to a water system in accordance with one or more embodiments. FIG. 13 displays several additional features, one or more of which can be implemented as a part of the THPS analyzer of the present application and its connection to a water system in accordance with one or more embodiments. As exemplified in FIG. 13, the solid and bubble issues discussed above were addressed by installing a filter 400 device up-side-down, an intermediate small vial 405, and a de-bubbler (not shown). More specifically, the incoming seawater goes through the filter device 400 to remove debris and most of the bubbles in the seawater line. The filters used in the filter device 400 used were GF/A Glass Microfiber Filter (Whatman Cat. No. 1820 047, 1.6 μm pore size, 47 mm diameter) and Ashless Grade 42 Quantitative Filter Paper (Whatman Cat. No. 1442 047, 2.5 μm pore size, 47 mm diameter), although it should be understood that other types of filters can be used in other embodiments.

The filter device 400 is placed in a way (i.e., upside-down) that the bubbles will stay above the filter and the majority of the seawater (along with bubbles) bypasses the filter and directly flows back to the drain 415 of the seawater sampling port. The seawater sample line from the bottom outlet of the filter device 400 goes to the small intermediate sample vial 405, from where a sample line is connected to the THPS analyzer via the 3-way valve 110. The setup of intermediate sample vial 405 further removes the bubbles in the seawater sample. A debubbler (100 μl capacity, −0.99 barg vacuum pump) was also installed between the reaction coil and the detector for removing the sporadic bubbles in the seawater stream and from the reaction of THPS and potassium permanganate.

With continued reference to FIG. 13, the flow rate of the seawater flowing through the THPS analyzer is controlled by a valve 420 in the main seawater line located behind the filter device 400. Closing this valve 420 pushes more seawater flowing through the analyzer. In the flow system of this experiment, a flow rate between 140 μl/min and 150 μl/min was desired, which is a bit challenging to achieve with the valve 420 in the main seawater line, without creating a backpressure on the sample intake port of the 6-way valve (not shown). As such, the intermediate sample vial 405 was used allowed sampling at atmospheric pressure, which eliminated the backpressure to the sample intake port of the 6-way valve, as well as further removing bubbles in the flow system.

The THPS analyzer was connected to the seawater shipping line with total dissolved solids (TDS) of seawater at 55-57 g/L. Salt precipitation from the seawater can clog almost all parts of the flow system if dried out. The heated reaction coil has a very small diameter. Its bending area, which creates a turbulent flow for the mixing of the sample and reagent, is prone to buildup of reaction product or precipitates from the seawater, causing clogging of flow system or reduced flow rate. The clogging issue was mitigated by proper maintenance and manual de-clogging procedures as needed. The maintenance includes washing the flow system with 1% of 20% triton-100 (or Tween-20) solution before the analyzer start-up and shutdown, followed by washing with distilled water.

In this implementation, measuring the sample flow rate (140-150 μl/min) and reagent injection rate (50 μl/stroke) was necessary before the analyzer start-up in order to determine if the analyzer had a clogging issue in the flow system. To manually de-clog, a syringe filled with triton or distilled water was used to remove the clogging materials in the flow system. If necessary, segment-to-segment de-clogging of the flow system is performed to pinpoint the exact clogging location.

The seawater pipeline was treated with two biocide products at the treatment plant, 93 km upstream of the installed THPS analyzer (see Table 12, above). The THPS-based biocide was planned to be injected every 2 weeks at 750 ppm for 90 minutes. However, there were deviations from the plan. For instance, some injections were delayed or cancelled due to operation issues or long holiday. In addition, the biocide treatment operation was challenged to maintain the planned injection dosage and time with outdated equipment and control system.

The THPS analyzer was installed in a field laboratory at the water supply plant and connected to the seawater shipping line for 29 weeks for the field demonstration. The THPS analyzer automatically measures BIOC31705A biocide residual concentrations when the biocide slug arrives at this location. From week 7 to 29, there were 9 biocide injections and 3 cancelled injections due to operation issues or long holiday (Table 13, below). Out of the 9 biocide injections, 7 biocide slugs were automatically detected by the THPS analyzer. One non-detection was due to the analyzer malfunction (i.e., tubing leak and bubbles in the flow system), and the other one is because the analyzer was used for other work and not connected to the seawater pipeline.

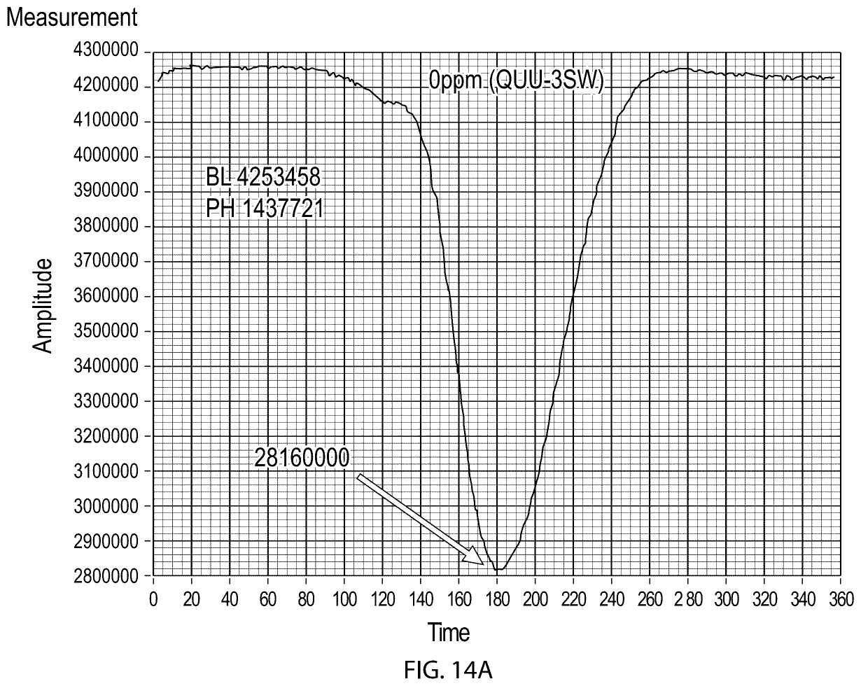

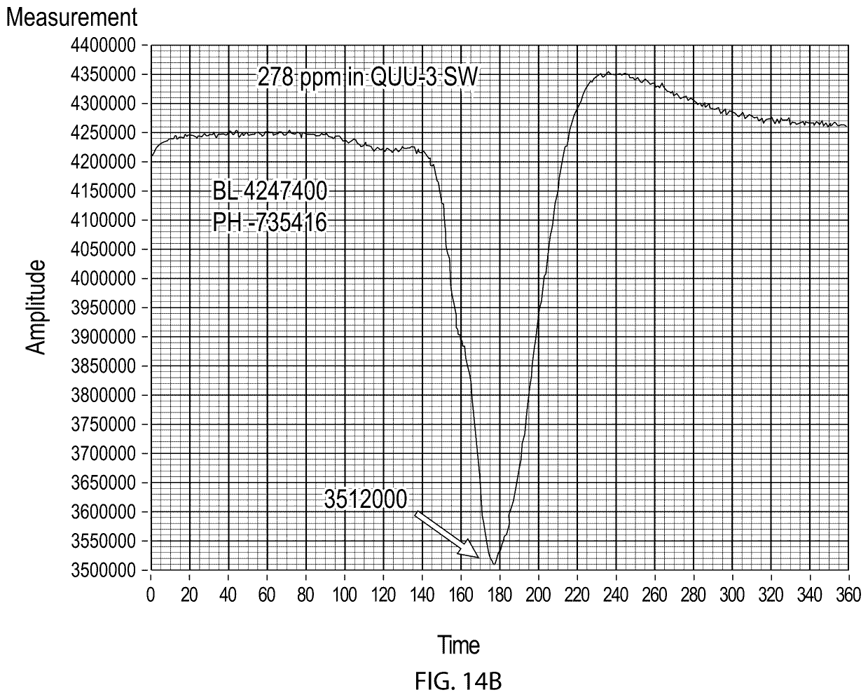

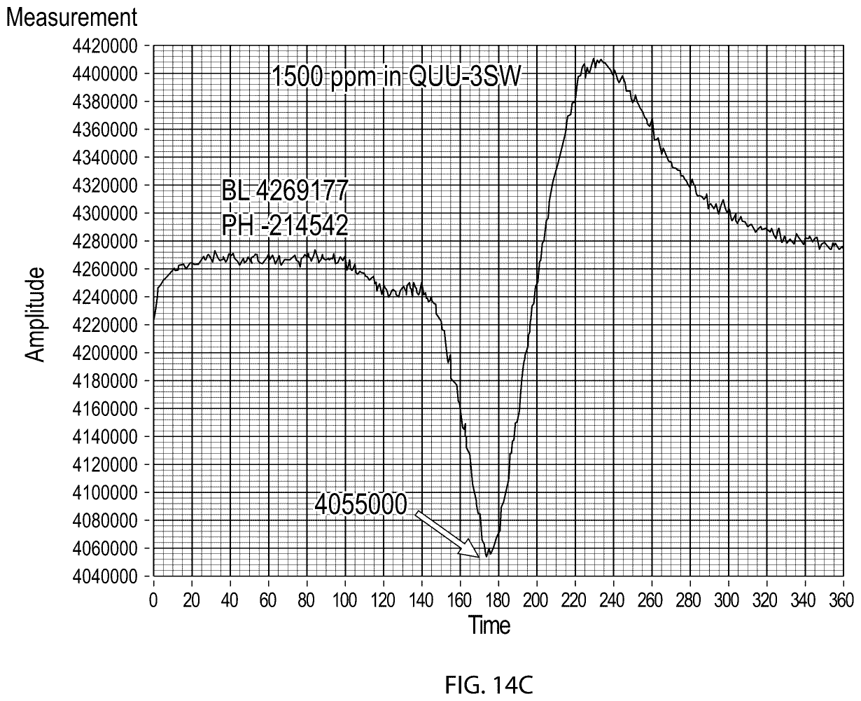

Three exemplary measurement curves for various concentrations of THPS biocide (BIOC31705A) are illustrated in FIGS. 14A-14C. Absorption peak height (PH) is the value of the lowest signal at the dip (absorption maximum) minus the baseline (BL) signal. As exemplified in FIGS. 14A-14C, the absolute value of the PH (dip depth) is inversely proportional to the concentration of THPS biocide in the sample, i.e., the lower the concentration, the bigger the dip.

TABLE 13
THPS biocide slug detection during field demonstration at a field laboratory.

| | | Injection | | | | | |
|---|---|---|---|---|---|---|---|
| Week | Date | time | | ETA at the analyzer | | | Remarks |
| 1-5 | Feb. 1, 2023 | 10:50 | AM | Feb. 2, 2023 | 6:27 | AM | Installation, operation, |
| | Feb. 14, 2023 | 1:00 | AM | Feb. 14, 2023 | 8:37 | PM | trouble-shooting and |
| | Feb. 27, 2023 | 10:00 | AM | Feb. 28, 2023 | 5:25 | AM | calibration |
| 7 | Mar. 13, 2023 | 10:00 | AM | Mar. 14, 2023 | 5:25 | AM | slug detected |
| 9 | Mar. 28, 2023 | 1:00 | AM | Mar. 28, 2023 | 8:51 | PM | slug detected |
| 11 | Apr. 10, 2023 | 9:00 | PM | Apr. 11, 2023 | 2:22 | PM | slug detected |
| 13 | May 1, 2023 | 9:00 | AM | May 2, 2023 | — | | Long holiday. No injection |
| | | | | | | | and measurement |
| 15 | May 15, 2023 | 9:00 | AM | May 16, 2023 | 7:39 | AM | Analyzer tubing leak and |
| | | | | | | | bubbles in the flow |
| | | | | | | | system. No measurement |
| 17 | May 29, 2023 | 10:00 | AM | May 30, 2023 | 9:42 | AM | slug detected |
| 19 | Jun. 19, 2023 | 9:00 | AM | Jun. 20, 2023 | 9:40 | AM | slug detected |
| 22 | Jul. 6, 2023 | 9:00 | AM | Jul. 7, 2023 | — | | Injection cancelled |
| 23 | Jul. 12, 2023 | 9:00 | AM | Jul. 13, 2023 | 10:47 | AM | The analyzer was used |
| | | | | | | | for other work. No |
| | | | | | | | measurement |
| 25 | Jul. 25, 2023 | 10:00 | AM | Jul. 26, 2023 | — | | QUU-3 pipeline isolated. |
| | | | | | | | No injection. |
| 27 | Aug. 8, 2023 | 10:00 | AM | Aug. 9, 2023 | 7:27 | AM | slug detected |
| 29 | Aug. 23, 2023 | 8:00 | AM | Aug. 24, 2023 | 8:22 | AM | slug detected |

Two biocide injections and the automated monitoring of biocide residual concentration at the water supple plant by the THPS analyzer are elaborated on below as examples.

### May 30, 2023 Slug

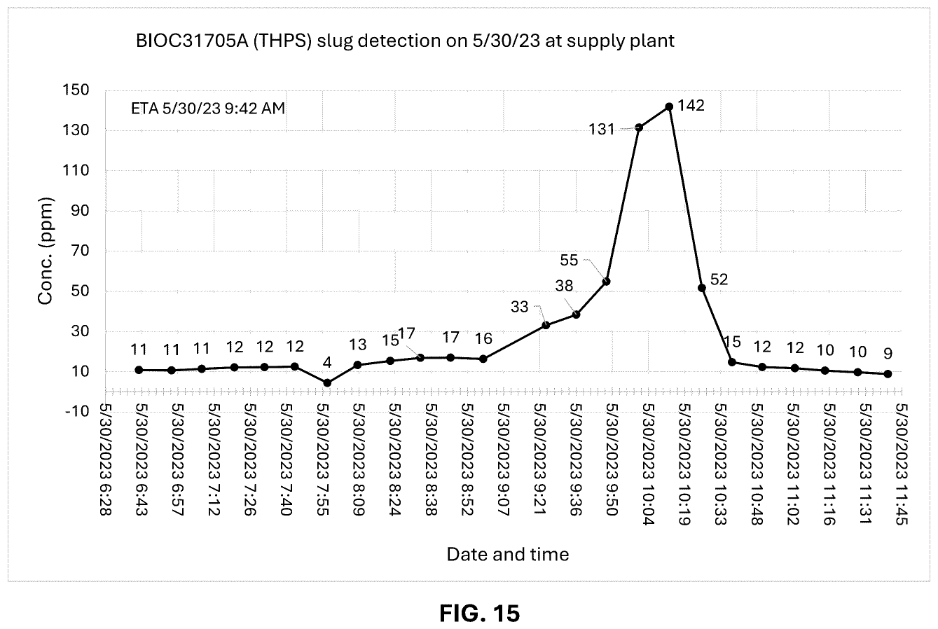

A THPS-based BIOC31705A biocide was injected at the sea water treatment plant on May 29, 2023, and the estimated time of arrival at the water supply plant was 9:42 am, May 30, 2023. The actual arrive time of the slug was around 9:20 am, and the slug passed the location at around 10:20 am, with the highest residual concentration detected at 142 ppm (see Table 14 and FIG. 15). The results indicated that the biocide was injected at much lower concentration than the planned dosage (750 ppm) at the seawater treatment plant (93 km upstream), and the injection duration was also shorter than planned (75 minutes). The THPS analyzer was calibrated at 0 ppm and 278 ppm of THPS biocide (2-point calibration) in the filtered seawater and using a 1.5 mM KMnO₄ reagent.

TABLE 14
THPS biocide slug measurement on
May 30, 2023 at the supply plant.

| Date and time | ppm | BL | PH |
|---|---|---|---|
| May 30, 2023 6:42 | 11 | 3899222 | −1205143 |
| May 30, 2023 6:55 | 11 | 3890714 | −1206704 |
| May 30, 2023 7:07 | 11 | 3878439 | −1200243 |
| May 30, 2023 7:20 | 12 | 3869983 | −1194177 |
| May 30, 2023 7:32 | 12 | 3860483 | −1193031 |
| May 30, 2023 7:44 | 12 | 3849845 | −1190831 |
| May 30, 2023 7:57 | 4 | 3841301 | −1262471 |
| May 30, 2023 8:09 | 13 | 3831381 | −1184505 |
| May 30, 2023 8:22 | 15 | 3822911 | −1167721 |
| May 30, 2023 8:34 | 17 | 3812943 | −1155569 |
| May 30, 2023 8:46 | 17 | 3819871 | −1155023 |
| May 30, 2023 8:59 | 16 | 3791896 | −1160068 |
| May 30, 2023 9:24 | 33 | 3850899 | −1041340 |
| May 30, 2023 9:36 | 38 | 3873769 | −1008898 |
| May 30, 2023 9:48 | 55 | 3932175 | −919294 |
| May 30, 2023 10:01 | 131 | 4047894 | −650418 |
| May 30, 2023 10:13 | 142 | 4107836 | −625731 |
| May 30, 2023 10:26 | 52 | 4122565 | −935188 |
| May 30, 2023 10:38 | 15 | 4125827 | −1173078 |
| May 30, 2023 10:50 | 12 | 4125852 | −1192536 |
| May 30, 2023 11:03 | 12 | 4125532 | −1197449 |
| May 30, 2023 11:15 | 10 | 4126928 | −1207871 |
| May 30, 2023 11:28 | 10 | 4126494 | −1214995 |
| May 30, 2023 11:40 | 9 | 4127085 | −1222492 |

### Aug. 9, 2023 Slug

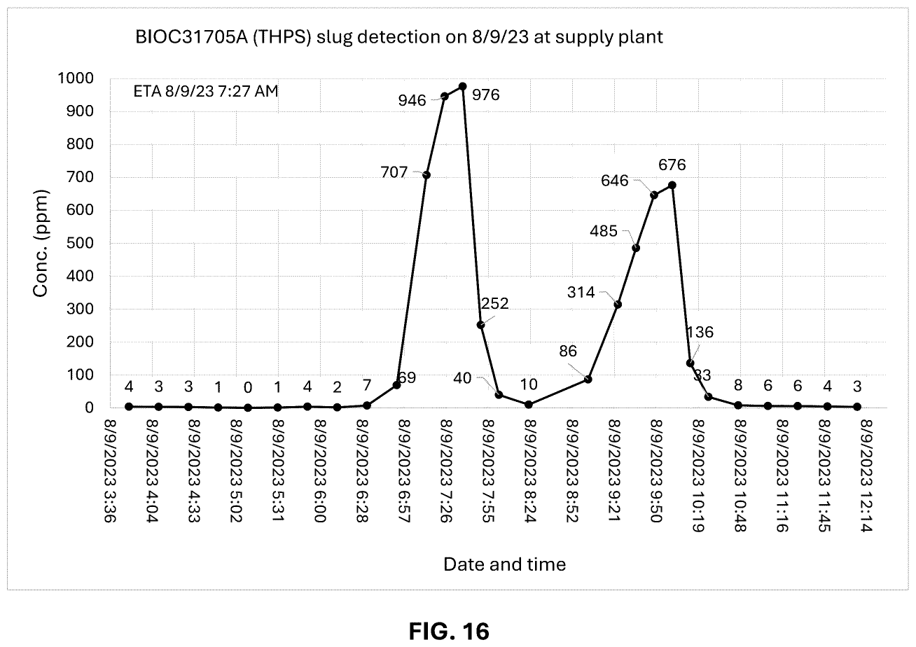

A THPS-based BIOC31705A biocide was injected at the sea water treatment plant on Aug. 8, 2023, and the estimated time of arrival at the water supply plant is 7:27 am, Aug. 9, 2023. The actual arrive time of the slug was around 7:00 am, and the slug passed the location at around 10:00 am (Table 15 and FIG. 16). In this injection, two biocide slugs were detected at water supply plant with approximately 1 hour gap, with the 1st slug at higher concentration (the highest residual concentration detected at 976 ppm). The total length of the two slugs was around 2 hours. The results indicated that the biocide was injected at higher than the planned dosage (750 ppm) at the treatment plant (93 km upstream), with a glitch in the injection operation. The analyzer was calibrated at 0 ppm and 278 ppm of THPS biocide (2-point calibration) in the filtered seawater and using 1.5 mM KMnO₄ reagent.

TABLE 15
THPS biocide slug measurement on
Aug. 9, 2023 at the supply plant.

| Date and time | ppm | BL | PH |
|---|---|---|---|
| Aug. 9, 2023 3:48 | 4 | 4240493 | −1285216 |
| Aug. 9, 2023 4:09 | 3 | 4243566 | −1287247 |
| Aug. 9, 2023 4:29 | 3 | 4243926 | −1290059 |
| Aug. 9, 2023 4:49 | 1 | 4247032 | −1298299 |
| Aug. 9, 2023 5:10 | 0 | 4247046 | −1303616 |
| Aug. 9, 2023 5:30 | 1 | 4248595 | −1299214 |
| Aug. 9, 2023 5:51 | 4 | 4248637 | −1285401 |
| Aug. 9, 2023 6:11 | 2 | 4249970 | −1296434 |
| Aug. 9, 2023 6:31 | 7 | 4251368 | −1266300 |
| Aug. 9, 2023 6:52 | 69 | 4240589 | −1014553 |
| Aug. 9, 2023 7:12 | 707 | 4246461 | −331987 |
| Aug. 9, 2023 7:25 | 946 | 4246854 | −265137 |
| Aug. 9, 2023 7:37 | 976 | 4246231 | −258539 |
| Aug. 9, 2023 7:49 | 252 | 4244546 | −638471 |
| Aug. 9, 2023 8:02 | 40 | 4245235 | −1120914 |
| Aug. 9, 2023 8:22 | 10 | 4244416 | −1253897 |
| Aug. 9, 2023 9:03 | 86 | 4237901 | −960600 |
| Aug. 9, 2023 9:23 | 314 | 4243694 | −567105 |
| Aug. 9, 2023 9:36 | 485 | 4245993 | −433187 |
| Aug. 9, 2023 9:48 | 646 | 4247334 | −354709 |
| Aug. 9, 2023 10:01 | 676 | 4245248 | −343128 |
| Aug. 9, 2023 10:13 | 136 | 4245677 | −834526 |
| Aug. 9, 2023 10:25 | 33 | 4247218 | −1146061 |
| Aug. 9, 2023 10:46 | 8 | 4248137 | −1263853 |
| Aug. 9, 2023 11:06 | 6 | 4248943 | −1273680 |
| Aug. 9, 2023 11:27 | 6 | 4245713 | −1274654 |
| Aug. 9, 2023 11:47 | 4 | 4245744 | −1282335 |
| Aug. 9, 2023 12:07 | 3 | 4242587 | −1288184 |

### Summary of Field Demonstration

Although many deviations from the plan were encountered during the field demonstration, in terms of injection interval, dosage, and duration, due to operation issues, outdated equipment and control system, the THPS analyzer successfully detected almost every biocide slug at the supply plant at the time around the estimated time of arrival.

The outcomes of the biocide slug detection during the field demonstration of prototype THPS analyzer were summarized in Table 16 below. The concentration of biocide slugs detected at this 93 km downstream location varied from 140 to 1000 ppm, with slug length varying from 1 to 3 hours. In 7 successful detections, 4 injections at the treatment plant produced 2 segments of biocide slugs at the water supply plant. The results from the field demonstration of the THPS analyzer indicate that the biocide injection dosage and duration at the treatment plant varied widely with frequent interruptions in injection operations.

TABLE 16
Summary of biocide slug detection at the supply
plant with prototype THPS analyzer.

| | THPS slug detection |
|---|---|
| No. of injection | 9 |
| No. of detection | 7 |
| No. of non-detection | 2 |
| analyzer related | 1 |
| non-analyzer related | 1 |
| No. of slug detected per injection | 1 or 2 |
| Slug duration | 1 to 3 hours |
| Max ppm of slug | 140 to 1000 ppm |

THPS Analyzer with Additional Features

In one or more embodiments, additional features can be added to the THPS analyzer—and in particular the aspects of the THPS analyzer that handles the liquid components—in order to further minimize certain technical deficiencies that can arise when the analyzer is implemented in certain environments. These deficiencies can include the need for additional bubble removal, flow rate (FR) fluctuations, backpressure, mixing reactor clogging, and varied mixing of the water sample and the reagent, for example.

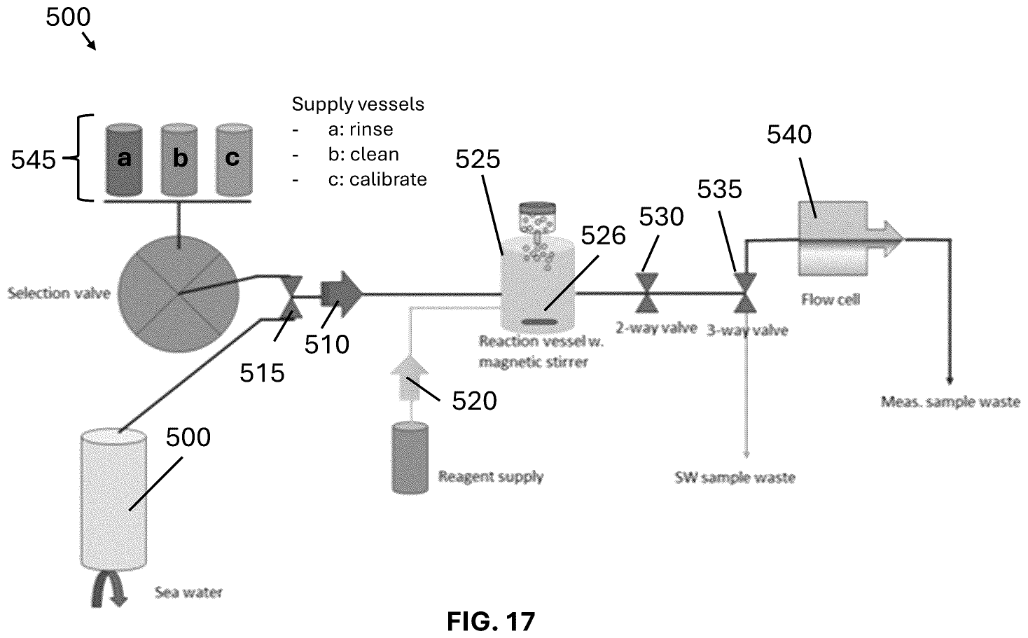

More specifically, in certain implementations large amount of bubbles can develop in the seawater line, which interfere with the measurement of absorption signals. Likewise, in certain implementations, backpressure can develop in the water sample line, causing the tubing carrying the water sample to pop out, leak, and decrease the flow rate of the sample, leading to quick wear-and-tear of the tubing. Regarding flow rate stability, in certain implementations, flow rate fluctuations can develop due to one or more factors, such as bubbles, clogging, backpressure, and tubing wear-and-tear. The peristaltic pump tubing can also require frequent replacement in certain implementations. Finally, in certain implementations, the reaction coil (e.g., a knitted coil) can become clogged, especially at a bending corner. The coil can become prone to solids buildup, causing flow rate decrease, and routine cleaning maintenance with triton and distilled water is not always sufficient to prevent or remove the clogging. In view of these issues that can arise in certain implementations, a THPS analyzer that includes one or more additional features as exemplified in FIG. 17 is provided.

Specifically, FIG. 17 displays a THPS analyzer 500 comprising additional features for the liquid handling aspects of the analyzer in accordance with one or more embodiments. In one or more embodiments, the THPS analyzer 500 can be implemented in a similar manner as the analyzer of FIGS. 1A-1C and 4A-4C, including being implemented in one or more cabinets, for example. With reference to FIG. 17, the THPS analyzer 500 can include a cross-filter 505 which is used to remove debris and most of bubbles. The cross filter 505 can be operatively connected to the water (e.g., seawater) that the water sample is collected from. Additionally, a piston pump 510 can be included to move the water sample continuously via a 2/3-way valve 515 through to the reaction vessel 525 and the rest of the analyzer 500. It should be understood that during a calibration cycle, the piston pump 510 moves the calibration solution same way as it moves the water sample through the analyzer. In one or more embodiments a metering pump 520 can also be included, where the reagent (KMnO₄) is injected via the metering pump 520 to a reaction vessel 525. The reaction vessel 525 in this embodiment replaces the reaction coil in the embodiments shown in FIGS. 1A-1C in implementations in which the reaction coil is prone to clogging, which can impact flow stability and measurement accuracy. In certain implementations, the reaction vessel provides 1) a consistent mixing of THPS from the water sample or calibration solution and the potassium permanganate reagent, 2) stable flow, 3) sampling at atmospheric pressure, and 4) bubble removal, thereby improving the measurement accuracy.

The water sample or calibration solution is mixed with the reagent at a reaction vessel, where the bubbles are further released. The reaction vessel 525, in certain embodiments, can include a magnetic stirrer 526 for hastening the reaction between the THPS in the water sample and the reagent. A downstream 2-way valve 530 can be closed while the water sample and the reagent are mixed in the reaction vessel.

After mixing, the mixed solution of the water sample and the reagent moves through the 2-way valve 530 (now opened) and the 3-way valve 535 to the detector 540 comprising the flow cell for absorption measurement of permanganate, which is correlated with the concentration of THPS in the water sample or calibration solution. The flow cell of the detector 540 operates in the same fashion as the flow cell described above with reference to FIGS. 1A-1C.

In the embodiment of the analyzer 500 in FIG. 17, the upside-down filter setup, intermediate sample vial, and de-bubbler as described in the embodiment of FIG. 13 are not included. These components were designed to remove debris and eliminate the large amount of the bubbles in the de-pressured water line at atmospheric sampling pressure. However, in the embodiment of the analyzer 500 of FIG. 17, the cross-flow filter 505 and the reaction vessel 525 with magnetic stirrer 526 achieve the same desired functionalities, and in certain implementations, can provide additional benefits.

Similarly, the piston pump 510 replaces the peristaltic pump and tubing as shown in the embodiments shown in FIGS. 1A-1C. In certain embodiments, the piston pump 510 can further improve the ruggedness and resilience of the analyzer.

Additionally, in one or more embodiments, the analyzer 500 can include an automated cleaning and rinsing feature 545 to further mitigate any clogging issues caused by solid buildup in the liquid handling aspects of the analyzer. The automated cleaning and rinsing cycles ease the required maintenance for the industrial analyzer.

Other modifications can be implemented across the various embodiments of the THPS analyzer of the present application. For example, the temperature of seawater in the shipping line varies widely daily and seasonally, which can affect baseline signals in different seasons, and therefore affects the calibration and measurement accuracy. Accordingly, in one or more embodiments, the analyzer includes an integrated cooling and heating mechanism to maintain a stable sample and analyzer cabinet temperature. The stable cabinet temperature can also improve the stability of optical and electronic systems. Similarly, the Z-flow cell, in certain embodiments can be made from Ultem® polyetherimide or polyether ether ketone (PEEK) polymer.

As discussed above, in one or more embodiments, the THPS analyzer of the present application can be operatively connected to a data processor. More specifically, in one or more embodiments the THPS analyzer disclosed herein can be part of or integrated with a computer implemented system, which can be configured with one or more data processors, memory, a controller, and a display, for example to process data received from connected devices into visual information.

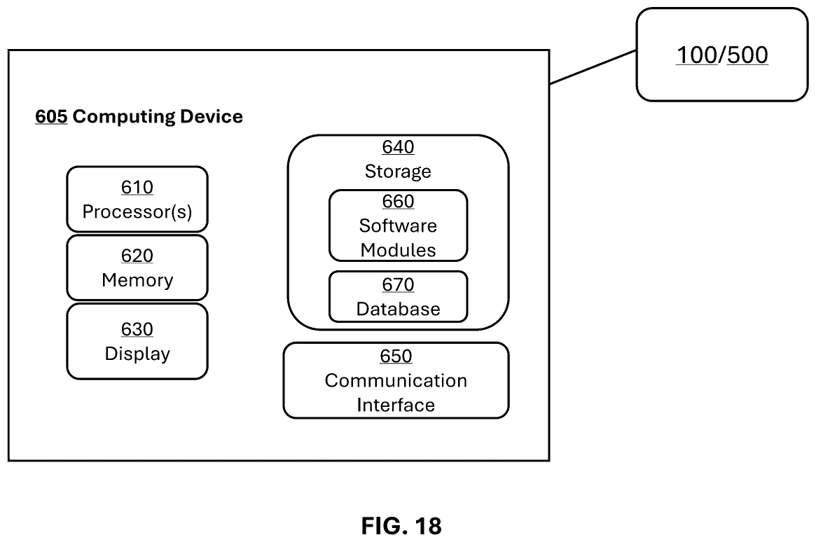

FIG. 18 is a block diagram illustrating an exemplary configuration of an exemplary computing device 605 of the system for determining the presence and concentration of THPS in a water sample, which can be operatively connected to a THPS analyzer 100/500 of the present application in accordance with one or more embodiments. The computing device 605 includes various hardware and software components that serve to enable operation of the system, including one or more data processors 610, a memory 620, a display 630, a storage 640 and a communication interface 650. Processor 610 serves to execute software instructions that can be loaded into memory 620. Processor 610 can be a number of processors, a multi-processor core, or some other type of processor, depending on the particular implementation.

Preferably, memory 620 and/or storage 640 are accessible by processor 610, thereby enabling the processor 610 to receive and execute instructions stored on memory and/or on storage. Memory can be, for example, a random access memory (RAM) or any other suitable volatile or non-volatile computer readable storage medium. In addition, memory can be fixed or removable. Storage 640 can take various forms, depending on the particular implementation. For example, storage can contain one or more components or devices such as a hard drive, a flash memory, a rewritable optical disk, a rewritable magnetic tape, or some combination of the above. Storage also can be fixed or removable.

One or more software modules, generally indicated at 660, can be encoded in storage 640 and/or in memory 620. The software modules can comprise one or more software programs or applications having computer program code or a set of instructions executed in processor 610. Such computer program code or instructions for carrying out operations for aspects of the systems and methods disclosed herein and can be written in any combination of one or more programming languages. The program code can execute entirely on the computer device 605, as a stand-alone software package, partly on computer device 605, or entirely on another computing/device or partly on another remote computing/device. In the latter scenario, the remote computing device can be connected to computer device 605 through any type of direct electronic connection or network, including a local area network (LAN) or a wide area network (WAN), or the connection can be made to an external computer (for example, through the Internet using an Internet Service Provider).

For example, included among the software modules can be a calibration module that receives and processes the data from a 2-point calibration procedure (e.g., calibration factors a and b) which can then be stored in memory for used during measurements of water samples with unknown amounts of THPS. Other modules can be provided to perform the functions described herein, such as temperature control of the heater; processing of each water sample, including the drawing in of each water sample and the disposal of each sample as waste after measurements have been conducted; a user interface module; etc. that are executed by processor 610. During execution of the software modules, the processor 610 configures the computer device 605 to perform various operations relating to the THPS analyzer.

It can also be said that the program code of software modules and one or more computer readable storage devices (such as memory and/or storage) form a computer program product that can be manufactured and/or distributed in accordance with the present invention, as is known to those of ordinary skill in the art.

It should be understood that in some illustrative embodiments, one or more of software modules can be downloaded over a network to storage from another device or system via communication interface for use within the system. In addition, it should be noted that other information and/or data relevant to the operation of the present systems and methods (such as database) can also be stored on storage, as will be discussed in greater detail below.

Also preferably stored on storage is database 670. The database contains and/or maintains various data items and elements that are utilized throughout the various operations of the system. The information stored in database can include but is not limited to, parameter adjustment algorithms, set-points, settings, alarms, actual values for process variables, and historical data collected and analyzed by the computer device 605. It should be noted that although database 670 is depicted as being configured locally to computer device 605 in certain implementations database and/or various of the data elements stored therein can be located remotely (such as on a remote computing device or server—not shown) and connected to computer device 605 through a network or in a manner known to those of ordinary skill in the art.

The communication interface 650 is also operatively connected to the processor 610. The interface can be one or more input device(s) such as switch(es), button(s), key(s), a touch-screen, microphone, etc. as would be understood in the art of electronic computing devices. The interface 650 serves to facilitate the capture of commands from the user such as on-off commands or settings related to operation of the analyzer.

The display 630 is also operatively connected to processor 10. Display 630 can include a screen or any other such presentation device which enables the user to view information relating to operation of the system including control settings, command prompts and data collected by various components of the system and provided to computer device 605. By way of further example, interface 650 and display 630 can be integrated into a touch screen display. Accordingly, the screen is used to show a graphical user interface, which can display various data and provide “forms” that include fields that allow for the entry of information by the user. Touching the touch screen at locations corresponding to the display of a graphical user interface allows the person to interact with the device to enter data, change settings, control functions, etc. So, when the touch screen is touched, interface 650 communicates this change to processor 610, and settings can be changed or user entered information can be captured and stored in the memory.

Communication interface 650 is operatively connected to the processor 610 and can be any interface that enables communication between the computer device 605 and external devices, machines and/or elements. Preferably, communication interface 650 includes, but is not limited to, Ethernet, IEEE 1394, parallel, PS/2, Serial, USB, VGA, DVI, SCSI, HDMI, a Network Interface Card (NIC), an integrated network interface, a radio frequency transmitter/receiver (e.g., Bluetooth, cellular, NFC), a satellite communication transmitter/receiver, an infrared port, and/or any other such interfaces for connecting computer device 605 to other computing devices and/or communication networks such as private networks and the Internet. Such connections can include a wired connection (e.g., using the RS232 standard) or a wireless connection (e.g., using the 802.11 standard) though it should be understood that communication interface can be practically any interface that enables communication to/from the computer device 605.

The processor(s) 610 can be configured, for example, by executing instructions stored on non-transitory processor readable media, to process information of various types and from various sources, including live visual data and data provided via one or more of sensors, instrument controllers, and display(s).

In one or more embodiments, the one or more processors can be configured by executing instructions, for example, provided in a series of software and/or hardware modules, to interpret, manipulate and record information received from the site at which each water sample is capture. Moreover, the processors can be configured to manipulate and provide illustrative and graphical overlays, and generate composite or hybrid visual data to the display device. The analyzer system can further include haptic technology that provides vibratory or other feedback in response to information processed by one or more processors.

Although much of the foregoing description has been directed to, the apparatus and methods disclosed herein can be similarly deployed and/or implemented in scenarios, situations, and settings far beyond the referenced scenarios. It should be further understood that any such implementation and/or deployment is within the scope of the methods described herein.

It is to be further understood that like numerals in the drawings represent like elements through the several figures, and that not all components and/or steps described and illustrated with reference to the figures are required for all embodiments or arrangements. Further, the terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms ““including,” “comprising,” or “having,” “containing,” “involving,” and variations thereof herein, when used in this specification, specify the presence of stated features, integers, steps, operations, elements, and/or components, but do not preclude the presence or addition of one or more other features, integers, steps, operations, elements, components, and/or groups thereof.

It should be noted that use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Notably, the figures and examples above are not meant to limit the scope of the present disclosure to a single implementation, as other implementations are possible by way of interchange of some or all the described or illustrated elements. Moreover, where certain elements of the present disclosure can be partially or fully implemented using known components, only those portions of such known components that are necessary for an understanding of the present disclosure are described, and detailed descriptions of other portions of such known components are omitted so as not to obscure the disclosure. In the present specification, an implementation showing a singular component should not necessarily be limited to other implementations including a plurality of the same component, and vice-versa, unless explicitly stated otherwise herein. Moreover, applicants do not intend for any term in the specification or claims to be ascribed an uncommon or special meaning unless explicitly set forth as such. Further, the present disclosure encompasses present and future known equivalents to the known components referred to herein byway of illustration.

The foregoing description of the specific implementations will so fully reveal the general nature of the disclosure that others can, by applying knowledge within the skill of the relevant art(s), readily modify and/or adapt for various applications such specific implementations, without undue experimentation, without departing from the general concept of the present disclosure. Such adaptations and modifications are therefore intended to be within the meaning and range of equivalents of the disclosed implementations, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s). It is to be understood that dimensions discussed or shown are drawings are shown accordingly to one example and other dimensions can be used without departing from the disclosure.

The subject matter described above is provided by way of illustration only and should not be construed as limiting. Various modifications and changes can be made to the subject matter described herein without following the example embodiments and applications illustrated and described, and without departing from the true spirit and scope of the invention encompassed by the present disclosure, which is defined by the set of recitations in the following claims and by structures and functions or steps which are equivalent to these recitations.

What is claimed is:

1. An analyzer for detecting and quantifying a tetrakis(hydroxymethyl)phosphonium sulfate (THPS) composition in a water sample, the analyzer comprising:
a peristaltic pump and a three-way valve operatively connected to the peristaltic pump, wherein the peristaltic pump is configured to draw in a water sample comprising a THPS composition through the 3-way valve;
a six-way valve operatively connected to the three-way valve via the peristaltic pump, wherein the six-way valve is configured to receive: a) the water sample from the 3-way valve via a first inlet and b) a potassium permanganate (KMnO₄) reagent via a second inlet;
a reaction coil operatively connected to the 6-way valve and configured to receive the water sample and the KMnO₄ reagent, wherein the water sample and the KMnO₄ reagent are mixed and reacted in the reaction coil;
a debubbler operatively connected to reaction coil and configured to receive the mixture of the water sample and the KMnO₄ reagent and remove bubbles from the mixture; and
a flow cell in a Z configuration comprising a 525 nm LED and a 650 nm LED, wherein the flow cell is configured to:
receive the mixture from the debubbler;
measure an intensity of KMnO₄ absorption in the mixture at a wavelength of 525 nm;
normalize the measured intensity by subtracting a background intensity at a wavelength of 650 nm; and
determine a presence and a concentration of the THPS composition in the water sample.

2. The analyzer of claim 1, wherein a correlation of the KMnO₄ absorption and the concentration of the THPS composition is based on the equation:
$$Y = \frac{1}{a + bX} ,$$
wherein Y is the peak height of absorption signal, X is the concentration of the THPS composition in the water sample, and a and b are calibration factors determined using a 2-point calibration procedure.

3. The analyzer of claim 2, wherein to perform the 2-point calibration procedure the flow cell is configured to:
measure an intensity of KMnO₄ absorption at a wavelength of 525 nm in a mixture of a water sample without the THPS composition and the KMnO₄ reagent to determine calibration factor a, wherein
$$a = \frac{1}{PH}$$
and PH is the peak height of the absorption signal; and
measure an intensity of KMnO₄ absorption at a wavelength of 525 nm in a mixture of a water sample with a known concentration c of the THPS composition and the KMnO₄ reagent to determine calibration factor b, wherein
$$b = \frac{\frac{1}{PH} - a}{c} .$$

4. The analyzer of claim 1, wherein the three-way valve comprises a water sample inlet for receiving the water sample, a calibration inlet for a calibration fluid, and an outlet 113 configured to direct the water sample or calibration fluid to the remaining components of the analyzer.

5. The analyzer of claim 1, further comprising a reagent injector configured to inject the potassium permanganate reagent into the 6-way valve.

6. The analyzer of claim 1, wherein the water sample is a calibration fluid, and wherein when the 3-way valve receives a calibration fluid, the analyzer is configured to perform a calibration cycle.

7. The analyzer of claim 1, wherein the analyzer has a dynamic detection range for the THPS composition of approximately 80-2000 ppm.

8. The analyzer of claim 1, wherein the THPS composition is a biocide composition and wherein the water is seawater.

9. The analyzer of claim 1, further comprising a heater operatively connected to the reaction coil, wherein the heater is configured to maintain the reaction coil at a temperature in the range of approximately 35-45° C.

10. The analyzer of claim 9, wherein the heater is configured to maintain the reaction coil at approximately 40° C.

11. The analyzer of claim 1, further comprising a cooling plate configured to cool a container comprising the KMnO₄ reagent, wherein the cooling plate is configured to maintain the container at a temperature in the range of approximately 2-8° C.

12. The analyzer of claim 1, wherein the measurement accuracy of the concentration of the THPS composition is within ±20% of the actual THPS concentration.

13. The analyzer of claim 1, wherein the analyzer is an online analyzer operatively connected to a water system and configured to perform real-time measurements of water samples from the water system.

14. The analyzer of claim 13, wherein the analyzer is configured to automatically switch from a slug mode to a non-slug mode if the measured concentration of the THPS composition is below a first predetermined value and to switch from non-slug mode to slug mode if the measured concentration of the THPS composition is above a second predetermined value.

15. The analyzer of claim 11, further comprising:
a filter device operatively connected to the water system via tubing that receives the water sample from the water system, wherein the filter device is configured to remove debris and bubbles from the water sample; and
an intermediate sample vial operatively connected to the three-way valve and configured to receive the water sample after passing through the filter device and further remove bubbles in the water sample,
wherein the filter device and the intermediate sample vial are located upstream of the three-way valve.

16. An online analyzer operatively connected to a water system for detecting and quantifying a tetrakis(hydroxymethyl)phosphonium sulfate (THPS) composition in a water sample, the analyzer comprising:
a cross-flow filter operatively connected to the water system via tubing that receives a water sample comprising a THPS composition from the water system, wherein the filter device is configured to remove debris and bubbles from the water sample;
a piston pump configured to draw in the water sample from the water system through the cross-flow filter;
a reaction vessel operatively connected to the piston pump and configured to receive the water sample via the piston pump and a potassium permanganate (KMnO₄) reagent from a reagent supply, wherein the water sample and the KMnO₄ reagent are mixed and reacted in the reaction vessel and the reaction vessel is further configured to remove bubbles from the water sample and KMnO₄ reagent mixture;
a flow cell in a Z configuration comprising a 525 nm LED and a 650 nm LED, wherein the flow cell is configured to:
receive the water sample and KMnO₄ reagent mixture from the reaction vessel;
measure an intensity of KMnO₄ absorption in the mixture at a wavelength of 525 nm;
normalize the measured intensity by subtracting a background intensity at a wavelength of 650 nm; and
determine a presence and a concentration of the THPS composition in the water sample.

17. The analyzer of claim 16, wherein a correlation of the KMnO₄ absorption and the concentration of the THPS composition is based on the equation:
$$Y = \frac{1}{a + bX} ,$$
wherein Y is the peak height of absorption signal, X is the concentration of the THPS composition in the water sample, and a and b are calibration factors determined using a 2-point calibration procedure.

18. The analyzer of claim 17, wherein to perform the 2-point calibration procedure the flow cell is configured to:
measure an intensity of KMnO₄ absorption at a wavelength of 525 nm in a mixture of a water sample without the THPS composition and the KMnO₄ reagent to determine calibration factor a, wherein
$$a = \frac{1}{PH}$$
and PH is the peak height of the absorption signal; and
measure an intensity of KMnO₄ absorption at a wavelength of 525 nm in a mixture of a water sample with a known concentration c of the THPS composition and the KMnO₄ reagent to determine calibration factor b, wherein
$$b = \frac{\frac{1}{PH} - a}{c} .$$

19. The analyzer of claim 16, wherein the analyzer has a dynamic detection range for the THPS composition of approximately 80-2000 ppm.

20. The analyzer of claim 16, wherein the measurement accuracy of the concentration of the THPS composition is within ±20% of the actual THPS concentration.
